(19) United States
(12) Patent Application Publication
(10) Pub. No.: US 2021/0318307 A1
(43) Pub. Date: Oct. 14, 2021

(54) PRECISION DIAGNOSIS OF CLOSTRIDIOIDES DIFFICILE INFECTION USING A SYSTEMS-BASED BIOMARKERS

(71) Applicant: Baylor College of Medicine, Houston, TX (US)

(72) Inventors: Tor Savidge, League City, TX (US); Qinglong Wu, Houston, TX (US)

(21) Appl. No.: 17/274,529

(22) PCT Filed: Sep. 19, 2019
(86) PCT No.: PCT/US2019/051950

§ 371 Date: Mar. 9, 2021

Related U.S. Application Data
(60) Provisional application No. 62/733,550, filed on Sep. 19, 2018.

(51) Int. Cl. G01N 33/569

(52) U.S. Cl. CPC G01N 33/56911; G01N 2333/195

(57) ABSTRACT

Embodiments of the disclosure include methods and compositions related to accurate diagnosis and treatment of medical conditions having diarrhea as a symptom. In specific cases, the disclosure concerns accurate assessment of a diarrheal cause related to the presence or risk that may or may not be a pathogenic infection, such as a Clostridioides difficule infection (CDI). Particular embodiments encompass one or more specific features that provide information for accurate diagnosis and treatment of CDI versus another cause for diarrhea.

### CROSS-REFERENCE TO RELATED APPLICATIONS

This application claims priority to U.S. Provisional Patent Application Serial No. 62/733550, filed Sep. 29, 2018, which is incorporated by reference herein in its entirety.

### STATEMENT REGARDING FEDERALLY SPONSORED RESEARCH OR DEVELOPMENT

This invention was made with government support under 5U01A1124290 awarded by National Institutes of Health. The government has certain rights in the invention.

### TECHNICAL FIELD

Embodiments of the field include bacteriology, cell biology, physiology, molecular biology, diagnostics, and medicine.

### BACKGROUND

Clostridioides difficule infection (CDI) is listed by the CDC as an urgent threat to public health. Early CDI diagnosis is crucial for optimal clinical management and improved prognosis. Due to the rapid turn-around and cost effectiveness, many hospitals utilize nucleic acid amplification tests to diagnose CDI. However, such sensitive molecular testing is widely recognized to misdiagnose up to 30% of CDI cases. A major reason for this misdiagnosis is that a positive stool test cannot differentiate Clostridioides difficule (formerly known as Clostridium difficule) colonization from symptomatic disease. Underscoring the importance of this assay deficiency, other factors including younger age and non-responsiveness to CDI therapy positively correlate with higher rates of alternative diagnoses, e.g., functional gastrointestinal disorders (FGIDs), inflammatory bowel disease (IBD), non-CDI infectious colitis. As such, there is an urgent need to generate a robust CDI diagnostic assay.

The present disclosure satisfies a long-felt need in the art of accurate CDI diagnosis and treatment.

### BRIEF SUMMARY

Given the risk for antimicrobial resistant (AMR)-pathogens causing life-threating infections, successful infectious disease management is critically dependent on identifying the most susceptible patient and determining the antibiotic susceptibility of the offending pathogen(s) to facilitate rapid clinical intervention. Although the value of precision infection management is well-recognized within the infectious disease community, neither the current analytical technology nor our understanding of host-pathogen risk associations is sufficiently well developed to initiate effective implementation.

The present disclosure is directed to methods and compositions that provide for accurate detection of C. difficule infection (CDI) in an individual. The methods can determine if an individual has CDI or does not have CDI. The methods can determine if an individual is at risk for CDI or is not at risk for CDI. Embodiments of the disclosure provide methods of identifying individuals that have CDI or are at risk for CDI (compared to age-matched or sex-matched individuals in the general population) and identifying individuals that do not have CDI or are not at risk for CDI (compared to the general population).

The individual may be of any kind, and the methods may be performed before, during, or after the individual has diarrhea. The methods may be performed when the individual is in need of antibiotics and/or antimicrobials of any kind or when the individual has already had antibiotics and/or antimicrobials of any kind. The methods may be performed as routine medical practice for an individual.

In some embodiments, the individual is a pediatric individual, and such an individual may or may not be a carrier of C. difficule. Pediatric individuals that are carriers of C. difficule would score positively for standard CDI assays (such as with 16S ribosomal RNA (rRNA)), but in methods of the disclosure they may be subjected to method steps that allow for determination of a cause of diarrhea that is not CDI. The pediatric individual may also be further defined as an individual that is less than about 4, 3, or 2 years of age, including an infant. The pediatric individual may be of an age in which the individual is not responsive to C. difficule toxins, and that individual may be assayed for and, in some cases, may be determined to have, diarrhea from a cause other than CDI. A pediatric individual may mature to the point that they become susceptible to CDI, and beyond that stage the individual may be subjected to methods encompassed herein to determine whether or not their diarrhea is from CDI.

In some embodiments, adults are subjected to methods of the disclosure to determine whether or not they have CDI. Adults generally are low risk for CDI unless they have taken an antibiotic and/or antimicrobial, including taken any antibiotic and/or antimicrobial at any time in their life or taken any antibiotic and/or antimicrobial within a certain time frame, such as within 10, 9, 8, 7, 6, 5, 4, 3, or 2 years, or within 1 year, or within 12, 11, 10, 9, 8, 7, 6, 5, 4, 3, or 2 months, or within 1 month, or within 4, 3, or 2 weeks, or within 1 week. An individual that has taken an antibiotic and/or antimicrobial is at greater risk for CDI than an individual that has not taken an antibiotic and/or antimicrobial, and this historical information for the individual may or may not be considered in determination of an outcome.

In some embodiments, an individual may not have diarrhea but is still subjected to analysis methods encompassed herein to determine an increased risk for having CDI. Any individual that is considered high risk for CDI may be provided a suitable treatment to prevent CDI, such as one or more antibiotics or prophylactic therapy including anti-virulence and/or microbial therapy. An individual may be determined to be a high risk individual based on the outcome of methods performed herein based on their genotype, family history, personal history, and overall health, including whether or not they already have a medical condition that may or may not be pathogenic infection and/or may or may not have diarrhea as a symptom. For example, as detailed in FIG. 8, an individual with a particular medical condition may be at high risk, moderate risk, or low risk for CDI. In one embodiment, an individual is high risk for CDI if they already have or have had antibiotic-associated diarrhea, acute myeloid leukemia, allogeneic hematopoietic stem cell transplantation, or have been in or are in an intensive care unit of a medical facility. Such an individual may or may not be provided a CDI treatment or prophylaxis. In another embodiment, an individual may be moderate risk for CDI if they already have or have had inflammatory bowel disease or cirrhosis. In a particular embodiment, an individual may be at low risk for CDI if they have or have had functional gastrointestinal disorders, metabolic syndrome, rheumatoid arthritis, or atherosclerosis.

Embodiments of the disclosure include prediction of patient susceptibility to a pathogen, such as C. difficule, by utilizing results from a systems-based data including fecal microbiome and metaproteome.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 illustrates a pattern affecting intestinal ecosystem with respect to antibiotic or antimicrobial use and CDI infection.

FIG. 2 shows colonization rates of toxigenic and nontoxigenic C. difficule in TEDDY cohort.

FIG. 3 shows microbiome signatures (top) and host signatures (bottom) for a CDI Index with respect to treatment with RBX2660. RBX2660 is an enema-administered microbiota-based treatment for the prevention of recurrent Clostridioides difficule infection.

FIG. 4 provides a schematic of full-length 16S rDNA, a species call for C. difficule amplicons of different length and 16S primer region, and an example of reproducible taxonomic compositions between control and CDI across different sequencing platforms, 16S primer regions in adults and children.

FIGS. 5A-5B (FIG. 5A) ROC curve analysis for supervised learning classifiers for adult training set (>1,200 cases). Classifiers build on the top 50 discriminative microbiome features and provides a significantly improved prediction of CDI diagnosis compared to other reported microbiota risk algorithms. (FIG. 5B) CDI patients harbor distinguishable gut microbiome features compared to healthy individuals. Denotation: pCDI, primary CDI; rCDI, recurrent CDI; AAD, antibiotic-associated diarrhea; FGID, functional GI disorders including IBS.

FIG. 6 demonstrates CDI risk during human development (TEDDY and American Gut cohorts).

FIG. 7 demonstrates CDI risk in human fecal microbiota bioreactors before and after antibiotic treatment. C. difficule invasion and colonization in bioreactors is only evident after antibiotic treatment when the CDI risk score is high.

FIGS. 8A-8B shows that the microbiome-based classifier provides population-scale measure of CDI risk index. (FIG. 8A) CDI risk index for general population enrolled in American Gut cohort (>10,000 subjects) is elevated with antibiotic use. (FIG. 8B) Adult microbiome-based classifier predicted CDI risk for hospitalized population (>5,000 patients).

FIGS. 9A-9B show that the microbiome-based classifier predicts FMT clinical outcomes in rCDI patients. (FIG. 9A) CDI risk classifier predicts the response of oral capsule-based FMT for adult recurrent CDI (rCDI) patients. The FMT donor CDI risk index is show to the right as healthy. (FIG. 9B) CDI risk classifier identifies the age difference in response to colonoscopy-based FMT for pediatric rCDI patients. The CDI risk index identifies pediatric FMR responders in older children with a diagnosis of recurrent CDI; children younger than 4 years who respond to FMT maintain a CDI high risk index and are likely misdiagnosed or asymptomatic carriers of C. difficule.

FIG. 10 provides an illustration of one embodiment of a multi-omics pipeline of metagenomics and metaproteomics feature generation for diagnosis of CDI patients.

FIG. 11 provides an illustration of one embodiment of a metaproteome method for high resolution mass spectrometry identification of functional features for diagnosis of CDI patients.

FIGS. 12 A and 12B illustrate microbiota community relative abundance and β diversity plots for 16S microbiome versus metaproteome generated signatures. (FIG. 12A) disparity of taxonomic composition between 16S-based profiling and metaproteomics-based profiling; (FIG. 12B) metaproteome features differentiate CDI from antibiotic-associated diarrhea (AAD), functional gastrointestinal disorders (FGID), and Control.

FIGS. 13A-13B provide ROC curve analyses for supervised learning classifiers for (FIG. 13A) WGS and (FIG. 13B) host metaproteome training sets. Classifiers build on the top 50 discriminative WGS microbiome or metaproteome features shows validation in fecal specimens from adult recurrent CDI patients treated with the microbiota-product RBX2660. RBX2660 is an enema-administered microbiota-based treatment for the prevention of recurrent Clostridioides difficule infection. Bottom panels show that host proteome features (FIG. 13B) provide a better classifier than WGS microbiome features for this treatment. Host metaproteome features also facilitate prediction of treatment outcome in baseline specimens before treatment with RBX2660.

FIGS. 14A-14C show protective microbiota features associated with CDI disease susceptibility. (FIG. 14A) Volcano plot showing the 50 most significant 16S features for diagnosis of CDI in patients. (FIG. 14B) Overlay assay showing antimicrobial activity of some microbiota example features targeting C. difficule VPI10463. (FIG. 14C) Quantitative data demonstrating statistically significant antimicrobial activity of some microbiota example features, two of which are not dependent on glycerol.

FIGS. 15A-15D demonstrate that CDI risk algorithm is broadly predictive of infection risk by diverse pathogens. (FIG. 15A) The microbiome features identify CDI development in a longitudinal cohort of patients with AML who underwent chemotherapy (red line); the microbiota classifier identifies patients at baseline who are at low risk of developing infection to CDI or any other pathogen (line in the bottom half of the image). A high risk index is also seen in patients at baseline who develop other infections (line in the top half of the image that begins lower than the other line). (FIG. 15B) The CDI risk classifier correctly predicts patients at low risk who do not develop clinical infection. The Inverse Simpson metric reflecting reduced a-diversity also trended lower in infected patients. Patients with a high risk index develop CDI, or local and system infection with the following pathogens:

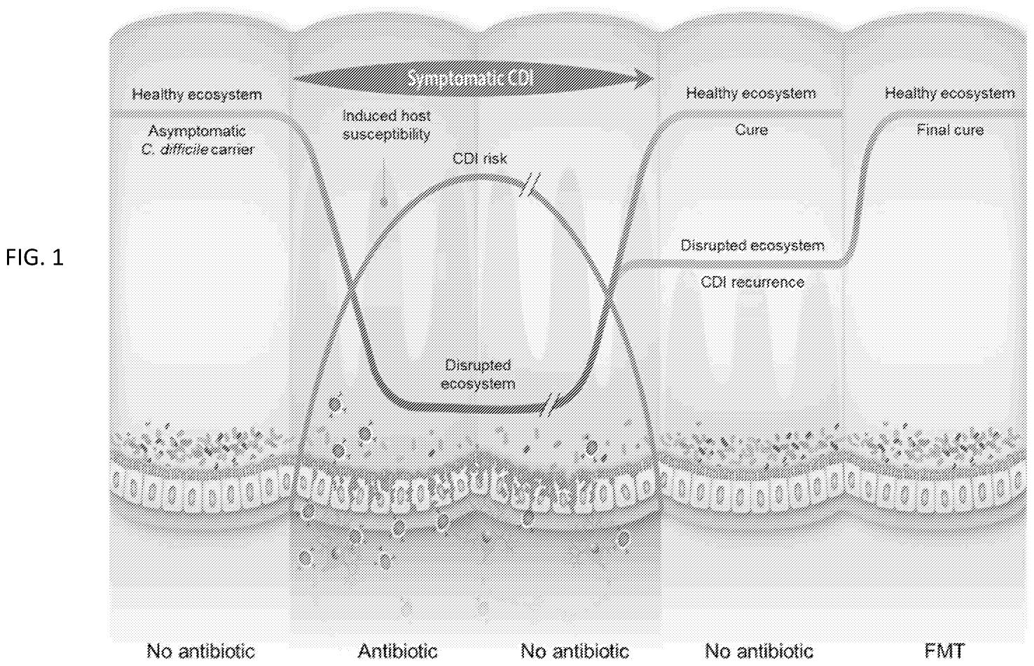

| | |
|---|---|
| Corynebacterium | Blood |
| Enterococcus faecium | Blood |
| Enterococcus | Urine |
| Escherichia coli | Urine |
| Escherichia coli | Blood |
| Fungal pneumonia | Sputum and throat swab |
| | sinusitis |
| Klebsiella | Blood |
| Pseudomonas areuginosa | Urine |
| Staphylococcus aureus (MRSA) | Upper respiratory tract |
| Stenotrophomonas pneumonia | Blood |
| Streptococcus pneumonia | Lung |
| Vancomycin-resistant Enterococcus | GI |

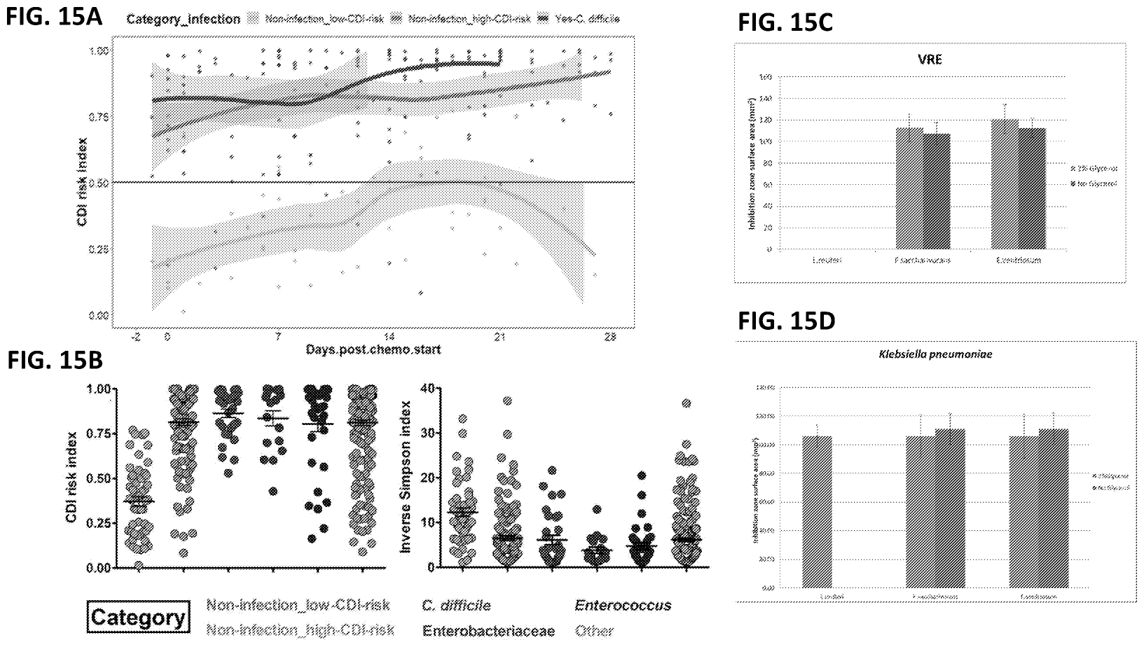

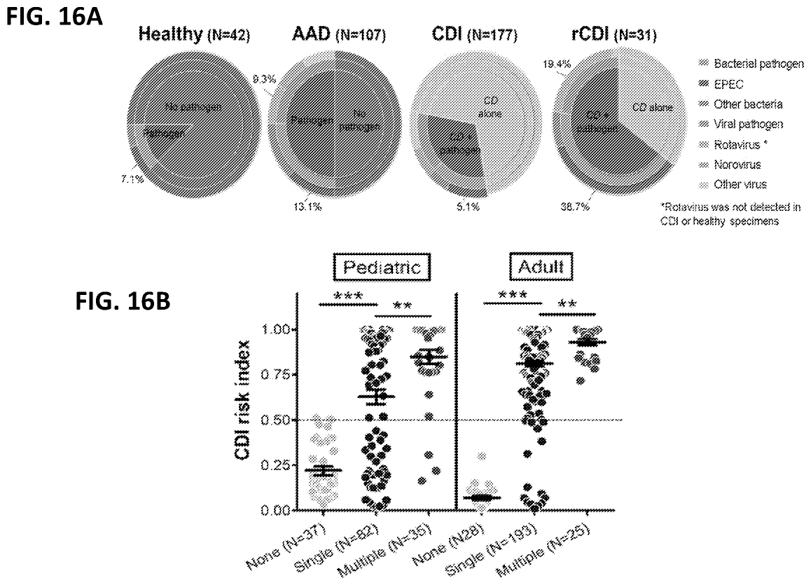

(FIGS. 15C and 15D) Quantitative data demonstrating statistically significant antimicrobial activity of some microbiota example features, two of which are not dependent on glycerol and show broad antimicrobial activity against VRE and Klebsiella pneumonieae.

FIGS. 16A-16B provide that risk classifier is associated with multiple pathogen detection by BioFire Film Array GI Panel. (FIG. 16A) Detection rate of 22 examples of pathogenic microbes in patients with CDI, recurrent CDI and AAD is shown and compared with healthy controls; Stool samples were tested with the FDA-approved BioFire FilmArray® GI Panel recognizing 12 bacteria: Campylobacter (jejuni, coli and upsaliensis), C. difficule, Plesiomonas shigelloides, Sal-monella, Yersinia enterocolitica, Vibrio (parahaemolyticus, vulnificus and cholerae), diarrheagenic E. coli/Shigella (enteroaggregative E. coli [EAEC], enteropathogenic E. coli [EPEC], enterotoxigenic E. coli [ETEC], Shiga toxin-producing E. coli [STEC] O157, and Shigella/Enteroinvasive E. coli [EIEC]); 4 parasites: Cryptosporidium, Cyclospora cayetanensis, Entamoeba histolytica, and Giardia lamblia; and 5 viruses: rotavirus A, adenovirus F 40/41, astrovirus, norovirus G1/GII, sapovirus I, II, IV, V). NIAID priority pathogens linked to the CDI algorithm also include patients with HIV, TB and malaria infection risk, but applies broadly to Clostridial infections and other infectious diseases. (FIG. 16B) Detection of multiple pathogens, including bacterial, viral and parasites in patients is predicted by a high CDI risk score (**, p<0.01; ***, p<0.001).

### DETAILED DESCRIPTION

### I. Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

The term “Antimicrobial” as used herein is a general term for drugs, chemicals, or other substances that either kill or slow the growth of microbes. Among the antimicrobial agents are antibacterial drugs, antiviral agents, antifungal agents, and antiparasitic drugs. In patients this includes drugs and/or treatment that impacts microbiome community composition.

As used herein, the terms “arrays”, “microarrays”, and “DNA chips” refer to an array of distinct oligonucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. The oligonucleotides on the array may be designed to bind or hybridize to specific nucleic acids, such as a specific SNP or a specific CNV, for example.

The terms “Clostridioides difficule infection” “C. difficule infection” or “CDI” as used herein refers to an individual that has presence of Clostridioides difficule in their body to an extent and under conditions in which a sufficient level of toxins from the Clostridioides difficule results in diarrhea. This is in contrast to presence of Clostridioides difficule in an individual that is considered a carrier for the bacteria and that has no diarrhea.

The term “classifier” as used herein refers to an algorithm that implements a disease classification, notably CDI diagnosis, or CDI risk or risk of C. difficule colonization. In other embodiments, the term refers to an algorithm that implements a disease classification for diagnosis or risk or risk of colonization for one or more pathogens other than C. difficule.

The term “feature” as used herein refers to a biological molecule that is representative of a detectable difference between a control or reference standard and the corresponding biological molecule in an individual with or at risk for CDI. The features may be nucleic acid (such as 16S rRNA), protein, small molecule, or a combination thereof.

As used herein, the term “oligonucleotide” refers to a short chain of nucleic acids, either RNA, DNA, and/or PNA. The length of the oligonucleotide could be less than 10 base pairs, or at minimum or no more than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49,50, 51, 52, 53, 54, 55, 56, 57, 58, 59, 60, 61, 62, 63, 64, 65, 66, 67, 68, 69, 70, 71, 72, 73, 74, or 75 base pairs. The oligonucleotide can be synthesized using by methods including phosphodiester synthesis, phosphotriester synthesis, phosphite triester synthesis, phosphoramidite synthesis, solid support synthesis, in vitro transcription, or any other method known in the art.

As used herein, the term “PCR primer” refers to an oligonucleotide that is used to amplify a strand of nucleic acid in a polymerase chain reaction (PCR). Primers may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 89%, 90%, 91%, 92%, 93%, 94%, 95%, 96%, 97%, 98%, 99%, or 100% homology to the template the primers hybridize to, wherein the 3′ nucleotide of the primer is complementary to the template. In some embodiments, lower annealing temperatures are used for initial cycles, for example cycles 1, 2, 3, 4, and/or 5, of the reaction.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

Clostridioides difficule, a common nosocomial pathogen, has been listed as top urgent threat to public health by CDC. C. difficule infection (CDI) after antibiotic therapy is effectively cured by fecal microbiota transplantation (FMT) by restoring heathy gut microbiota. Medications and therapy that disrupt gut microbiota are well recognized CDI risk factors supporting the concept that microbiota health is a key determinant in patient susceptibility to C. difficule. Although testing for microbiota susceptibility to CDI is evolving, it is still poorly developed. Embodiments of the disclosure provide methods and compositions related to guidelines for suitability of treatment for Clostridioides difficule infection.

In addition, CDI in children and adults is often associated with detection of other enteric pathogens. Indeed, by screening for 22 enteric pathogens using the FDA-approved BioFire Film Array Gastrointestinal (GI) Panel in a cohort of 356 children (age >3 yrs) with CDI or antibiotic-associated diarrhea (AAD), certain embodiments herein indicate that diverse enteric bacterial and viral pathogen colonization and/or infection is more common in children with a perturbed gut microbiota than in healthy controls (based on ROMEIII criteria) who have a normal microbiota community structure. Adults with a perturbed gut microbiota are also at higher risk of diverse enteric pathogen colonization and this correlates significantly with our CDI risk algorithm. Although lacking the specificity and sensitivity of PCR, the fecal metagenomics analysis of the disclosure is also supportive of co-colonization in CDI patients that indicate that the gut acts as a septic resevoir for other pathogens, and this pattern is reversed by FMT. The detection of diverse bacterial and viral pathogens in patients with dysbiosis promoted the inventors to test whether a CDI risk algorithm is universally predictive of infection risk in hospitalized patients. The inventors analyzed the longitudinal microbiome data of adult acute myeloid leukemia (AML) patients (N=105) who underwent chemotherapy at MD Anderson Cancer Center, Houston, and who were prospectively monitored for infection because this is a high occurrence in this patient population (≈40%): the inventors stratified infection diagnosis with a CDI risk algorithm and demonstrated a highly significant correlation. Reduced microbiome diversity is reported to be associated with infection risk and disclosure embodiments support this trend; however, the disclosure significantly advances this field by identifying new and previously untested candidate keystone microbiota species that are shown to be predictive of infection susceptibility by diverse pathogens. It also shown herein that at least some of these microbiota features demonstrate potent antimicrobial activity in overlay assays against multiple pathogens, including C. difficule, VRE and K. pneumonia.

### II. Methods of Use for Clostridioides Embodiments

Particular embodiments of the present disclosure concern methods, systems, and compositions for the diagnosis of, or prediction for, one or more diarrheal diseases in an individual. The diarrheal disease may be any disease that encompasses symptomatic diarrhea including, for example, antibiotic-associated diarrhea, a Clostridioides infection, or any functional gastrointestinal disease. The individual may be an adult, child, or infant.

Particular methods, systems, and compositions of the disclosure measure features in a sample from an individual. The sample may be a gastrointestinal sample including, for example, a gut sample, a fecal sample, or other samples collected from the gastrointestinal tract of the individual. The detection, or lack of detection, of specific features, in a certain combination, may indicate the individual has, or is likely to have, a Clostridioides infection. In some embodiments, the detection, or lack of detection of specific features, in a certain combination, may indicate the individual has, or is likely to have at least one recurrent Clostridioides infection. The detection, or lack of detection of other specific features, in a certain combination, may indicate the individual has, or is likely to have, antibiotic-associated diarrhea (AAD). The detection, or lack of detection, of specific features in specific combinations may indicate the individual has a diarrheal disease, including the diseases disclosed herein. Features for a specific disease may be different between different populations of individuals. For example the detection, or lack of detection, of specific features in a sample of an adult may indicate an adult has a Clostridioides infection, however the detection, or lack of detection, of the same specific features in the sample of a child may or may not indicate a child has a Clostridioides infection.

In particular embodiments, the levels and/or concentrations of detected features is further compared to a known standard, wherein comparison to a known standard indicates the individual as having or not having a diarrheal disease, including a Clostridioides infection, AAD, an FGID, or other diarrheal diseases disclosed herein. The levels and/or concentrations of features detected in methods of particular embodiments may be measured against known standard levels to indicate the individual has or does not have a Clostridioides infection, including a potentially recurring Clostridioides infection. The levels and/or concentrations of features detected in methods of particular embodiments may be measured against known standard levels to indicate the individual has or does not have AAD. The levels and/or concentrations of features detected in methods of particular embodiments may be measured against known standard levels to indicate the individual has or does not have an FGID.

In some embodiments, there may be one or more features that, when detected or not detected in a sample, are indicative of more than one diarrheal disease. In particular embodiments of the disclosure, the combination of detection, or lack of detection, of specific features in a sample from an individual indicates the individual has, or is likely to have, a specific diarrheal disease, including those disclosed herein. In some embodiments, the number of indicative features, either detected or not detected in a sample from an individual is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 1443, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 82, 183, 184, 185, 186, 187, 188, 189, 190, 191, 191, 192, 193, 194, 195, 196, 197, 198, 199, or 200 or more features encompassed herein for detecting a diarrheal disease, such as those disclosed herein. In particular embodiments, the number of indicative features, either detected or not detected in a sample from an individual is 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% features encompassed herein for detecting a diarrheal disease, such as those disclosed herein.

Particular embodiments of the disclosure concern the detection of features associated with a cellular and/or molecular response from an individual to microbiome species in the gastrointestinal tract of the individual, also known as a host response. Measuring the host response may allow for high predictively diagnosis and prognosis.

In particular embodiments, features include data collected from a sample, such as a gut, fecal, or other gastrointestinal sample. Data for identifying features as described herein may be from sequencing data, including 16S rDNA. 16S rDNA data may be used to determine the bacterial genus or species present in the sample. Data for identifying features as described herein may be from metabolomics data. Data for identifying features as described herein may be from proteomics data, which may include proteins expressed by the individual and/or proteins expressed by the microbiome located in the gastrointestinal tract of the individual.

Particular embodiments of the disclosure concern systems for measuring features from a sample, such as a gut, fecal, or other gastrointestinal sample. In particular embodiments, the system comprises one or more substrates that have molecules directly or indirectly representative of the presence of one or more features from a sample from an individual.

In particular embodiments, when the detection and/or measurement of specific features indicate an individual as having or not having a certain diarrheal disease, including a Clostridioides infection, AAD, an FGID, or other diarrheal diseases disclosed herein, the individual may be administered a therapy to treat the individual. The therapy may be at least one of an antibiotic, a curative therapy, and/or a symptom relief therapy. In particular embodiments, wherein an individual is indicated to have AAD and at the time of AAD diagnosis is on an antibiotic regimen, the administration of antibiotics may be stopped, or tapered off, to reduce the cause of diarrhea, wherein the reduction of the antibiotic is a method of treatment.

Particular embodiments employ a systems-based approach to identify microbiota and host biomarkers that differentiate CDI cases from antibiotic-associated diarrhea (AAD) and functional gastrointestinal diseases (FGIDs). Methods, systems, and compositions encompassed in particular embodiments employ supervised learning features based on systems data generated from >2,500 fecal microbiome (16S rDNA), metaproteome, metabolome, and clinical metadata profiles from adult and pediatric cases with CDI, AAD or FGID, and control subjects without GI disease. In some aspects, CDI-classification based on fecal 16S microbiome alone data may only provide >90% diagnostic accuracy, whereas classification accuracy may improve to >99% when adding metaproteome, metabolite, and/or clinical metadata features. Importantly, these improved features confidently distinguishing CDI from potential AAD and FGID misdiagnosis. In particular embodiments, supervised learning classification of systems-based metadata offers precision diagnosis of CDI versus non-infectious enteric disease at a population scale level.

In particular embodiments, a sample is obtained from an individual suffering from symptoms of diarrhea, including acute or chronic diarrhea. The sample may be any biological sample, including any sample from the gastrointestinal tract of the individual such as a fecal sample. Levels of features, which may include nucleic acids, metabolites, proteins, clinical metadata, or other quantifiable aspects of the sample, may be measured from the sample using methods practiced by the skilled artisan. The measured levels may be analyzed, such as by applying machine learning algorithms.

In certain embodiments, the methods and systems of analyzing features utilize a so-called training set of samples from individuals with known disease states or prognoses. For example, a training set with patients known to have or not have a CDI may be used. Once established, the training data set serves as a basis, model, or template against which the features, such as features disclosed herein, of an unknown sample from an individual are compared, in order to diagnose the individual with having or not having a disease or provide a prognosis of the disease state in the individual.

Embodiments of the disclosure include methods of determining a cause of diarrhea in an individual that is suffering from diarrhea, including recurrent diarrhea. In cases wherein the diarrhea is recurrent diarrhea, a sample may be taken from an individual during a bout of diarrhea or between bouts of diarrhea. The methods of determining a cause of diarrhea comprise measuring for one or more features in one or more of Tables A-C from a gut sample from the individual, including at least a fecal sample. In some cases, the individual has two or more causes of diarrhea. Following measurement of the one or more features of one or more of Tables A-C, a treatment regimen may be determined. The treatment regimen may be effective only because the measurement of the one or more features in one or more of Tables A-C was determined. In at least some cases, were it not for the measurement of the one or more features in one or more of Tables A-C, the individual would be administered an ineffective treatment that may or may not be harmful to the individual. The treatment regimen may or may not be modulated following measurement of the one or more features in one or more of Tables A-C. In some cases, the measurement allows for confirmation of an intended treatment. In specific embodiments, the methods further comprise modulating a treatment for the individual determined to have one or more features that indicate the presence or absence of one or more conditions (or treatments therefor) that result in diarrhea. In specific embodiments, the method further comprises administering a treatment or reducing a treatment to the individual when the individual is determined to have one or more features that indicate the presence or absence of one or more diarrheal-associated diseases. In specific embodiments, the individual having one or more particular features in one or more of Tables A-C is determined to have a Clostridioides infection, including at least of Clostridioides difficule, Clostridioides perfingens, Clostridioides botulinum, or a mixture thereof. In specific embodiments, the individual having one or more particular features is determined to have antibiotic-associated diarrhea and, in at least some cases, the antibiotic is halted or reduced in dosage following such determination.

Any method encompassed herein may utilize measuring of one or more features disclosed herein. The measuring in at least some cases identifies the presence or absence of one or more features encompassed in the disclosure herein. In some cases, the measuring identifies a level of one or more features encompassed in the disclosure herein, including a level that is compared to a threshold or known standard. Any suitable control, threshold or known standard may be utilized, but in specific embodiments the threshold or known standard is a reference from age-matched and/or sex-matched individuals who do not have diarrhea or do not have recurrent diarrhea.

Any mammalian individual susceptible to toxins of C. difficule may be subject to methods of the disclosure. The individual may be of any gender or age, including an adult, child, or infant. However, in specific embodiments, the individual is of a sufficient age to be susceptible to toxins of C. difficule, including at least or at least about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months of age. The individual may or may not have recurrent diarrhea. The individual may or may not be suspected of having misdiagnosis of a cause for any diarrhea, including recurrent diarrhea. The individual may be subject to methods of the disclosure to avoid having a misdiagnosis of a cause for any diarrhea, including recurrent diarrhea.

Methods of the disclosure include methods of treating an individual having diarrhea (recurrent or not) comprising measuring for one or more features encompassed in one or more of Tables A-C from a gut sample (including fecal sample) from the individual; and either (1) reducing the administration of one or more antibiotics to the individual when the individual has presence or absence or a certain level of one or more feature(s) encompassed in one or more of Tables A-C, for example said features being indicative of antibiotic associated diarrhea; or (2) administering one or more antibiotics and/or antimicrobials to the individual when the individual has presence or absence or a certain level of one or more feature(s) indicative of Clostridioides infection, for example said features being indicative of Clostridioides infection.

Methods of the disclosure include methods of treating an individual having diarrhea (recurrent or not) comprising measuring for one or more features encompassed in one or more of Tables A-C from a gut sample (including fecal sample) from the individual; and either (1) reducing the administration of one or more antibiotics for an individual determined to have the presence or absence or a certain level of one or more feature(s) encompassed in one or more of Tables A-C, for example said one or more features being indicative of antibiotic associated diarrhea; or (2) administering one or more antibiotics to an individual determined to have the presence or absence or a certain level of one or more feature(s) encompassed in one or more of Tables A-C, for example said features being indicative of Clostridioides infection.

Any antibiotics and/or antimicrobials to be provided to the individual when appropriate or to be avoided for the individual when appropriate may comprise at least one of the antibiotics and/or antimicrobials selected from the group consisting of a small molecule antibiotic, an antibiotic derived from a natural product, a microbial composition, an antibody or therapeutic suitable for neutralizing Clostridioides infections, and a combination thereof.

Embodiments of the disclosure include methods of measuring one or more features encompassed herein in a fecal or gut sample from an individual that has diarrhea, that has recurrent diarrhea, and/or that is suspected of having a misdiagnosis of a diarrheal cause, comprising the steps of two or more of the following: analyzing one or more nucleic acids in the sample; analyzing one or more metabolites in the sample; and analyzing one or more proteins in the sample. In specific embodiments, the analyzing includes analyzing for the presence and/or level of one or more features encompassed in one or more of Tables A-C. In cases wherein the nucleic acid from a sample is analyzed, the nucleic acid may be analyzed by sequencing, polymerase chain reaction, isothermal amplification, bioinformatics, or a combination thereof. The nucleic acid may be of any kind that is indicative of presence of Clostridioides, such as 16S ribosomal RNA. Any nucleic acid analysis may or may not include whole genome sequencing, yet in specific cases it does not include whole genome sequencing. In cases wherein metabolites from a sample are analyzed, the analysis may be by mass spectrometry, ELISA, chromatography, or a combination thereof. In cases wherein proteins are analyzed from a sample, the proteins may be analyzed by mass spectrometry, ELISA, chromatography, Western blotting, immunoprecipitation, immunoelectrophoresis, or a combination thereof.

Embodiments of the disclosure include methods to measure a host response to a microbial infection in an individual, said individual that has diarrhea, that has recurrent diarrhea, and/or that is suspected of having a misdiagnosis of a diarrheal cause, comprising the steps of analyzing one or more nucleic acids in a fecal or gut sample from the individual; analyzing metabolites in the sample; and/or analyzing proteins in the sample. In such methods, the microbial infection may be of any kind that causes diarrhea in a host, but in specific embodiments the infection is any species of Clostridioides that can cause diarrhea in a host. In such methods, the one or more features are encompassed in one or more of Tables A-C.

In particular embodiments of the disclosure, one identifies whether or not an individual is high risk, moderate risk or low risk of having CDI. Such embodiments include the ability to predict an outcome for the individual. Any analysis for any method herein may occur at the time that an individual has diarrhea, at the time or after that an individual has a second or subsequent bout of diarrhea, or as part of routine screening for general health purposes.

In specific embodiments, an individual is not subject to methods of disclosure unless they have had antibiotics and/or antimicrobials, given that generally healthy adults have a low risk of CDI unless they take antibiotics. Therefore, in specific embodiments a sample from an individual is measured for one or more feature(s) as encompassed herein before antibiotics and/or antimicrobials are administered, while antibiotics and/or antimicrobials are being administered, and/or after antibiotics and/or antimicrobials have been administered. The course of antibiotics or any antimicrobial treatment including chemotherapy may be a first exposure for the individual, although in some cases it is a second or subsequent exposure to antibiotics.

In particular methods of the disclosure, individuals with or at risk for CDI are able to be distinguished from individuals with our at risk for irritable bowel syndrome (IBS). In some cases, an individual with a first or subsequent bout of diarrhea is subjected to methods of the disclosure in which case one or more particular features identify an individual with or at risk for CDI or not as having or at risk for CDI. In some cases, CDI may be ruled out as a cause or risk for the individual and the individual is then determined whether or not they have IBS, whether or not that IBS determination utilizes information from feature(s) of the disclosure.

In pediatric individuals, some are of an early enough age that they are not yet susceptible to toxins from C. difficule, and yet they may be subjected to methods of the disclosure to determine their risk of CDI once they become old enough to be susceptible to the toxins. In some cases, the individual is not subjected to methods of the disclosure until they are suspected or shown to be susceptible to the toxins, for example suspected because they reach a certain age. Any of such screening methods may be performed as routine health care for the pediatric individual.

Embodiments of the disclosure allow for distinguishing whether or not features for an individual are suitable for indicating the presence or risk for CDI. In specific cases, the form of features that are analyzed needs to be indicative of the presence of live bacteria capable of producing toxins that cause diarrhea as opposed to dead bacteria that cannot. Therefore, in at least some cases one or more features that are used are not nucleic acid in form because nucleic acids may originate from dead bacteria. In specific cases, one or more non-nucleic acid features that represent metabolic activity are utilized to identify the presence of live bacteria that may be causing diarrhea, such as metabolites that may be small molecules and/or proteins.

Embodiments of the disclosure encompass methods wherein outcome of a therapy for CDI patients, including recurrent CDI, is predictable or determined based on the measurement of one or more features from one or more of Tables A-C. The therapy may be of any kind, including at least FMT, antibiotics, therapeutics, contact isolation, or a combination thereof.

Methods and compositions of the disclosure can distinguish an individual that has irritable bowel syndrome (IBS) versus an individual that has CDI. In specific cases, an individual having certain one or more features from one or more of Tables A-C is determined to have IBS instead of CDI, and in specific embodiments following this determination the individual is accurately treated for IBS instead of CDI. In other cases, an individual having certain one or more features from one or more of Tables A-C is determined to have CDI instead of IBS, and in specific embodiments following this determination the individual is accurately treated for CDI instead of IBS.

Methods and compositions of the disclosure can distinguish an individual that has antibiotic-associated diarrhea versus an individual that has CDI. In specific cases, an individual having certain one or more features from one or more of Tables A-C is determined to have antibiotic-associated diarrhea instead of CDI, and in specific embodiments following this determination the individual is accurately treated for antibiotic-associated diarrhea instead of CDI. In other cases, an individual having certain one or more features from one or more of Tables A-C is determined to have CDI instead of antibiotic-associated diarrhea, and in specific embodiments following this determination the individual is accurately treated for CDI instead of antibiotic-associated diarrhea.

### III. Methods of Use for Other Pathogenic Embodiments

Any of the embodiments encompassed herein related to Clostridioides may be applied to any other pathogen of any kind, including the specific features encompassed in Tables A-C. The pathogen may be a bacteria, virus, parasite, fungus, or combination thereof. In specific cases, the pathogen is one or more of the following: Campylobacter (jejuni, coli and/or upsaliensis); C. difficule; Plesiomonas shigelloides; Salmonella; Yersinia enterocolitica; Vibrio (parahaemolyticus, vulnificus and/or cholerae); diarrheagenic E. coli/Shigella (enteroaggregative E. coli [EAEC]; enteropathogenic E. coli [EPEC]; enterotoxigenic E. coli [ETEC]; Shiga toxin-producing E. coli [STEC] O157; Shigella/Enteroinvasive E. coli [EIEC]); Cryptosporidium; Cyclospora cayetanensis; Entamoeba histolytica; Giardia lamblia; rotavirus A; adenovirus F 40/41; astrovirus; norovirus Gl/GII; sapovirus I, II, IV, and/or V

Particular embodiments of the present disclosure concern methods, systems, and compositions for the diagnosis of, or prediction for, one or more diarrheal diseases in an individual. The diarrheal disease may be any disease that encompasses symptomatic diarrhea including, for example, antibiotic-associated diarrhea, a pathogenic infection, or any functional gastrointestinal disease. The individual may be an adult, child, or infant.

Particular methods, systems, and compositions of the disclosure measure features in a sample from an individual. The sample may be a gastrointestinal sample including, for example, a gut sample, a fecal sample, or other samples collected from the gastrointestinal tract of the individual. The detection, or lack of detection, of specific features, in a certain combination, may indicate the individual has, or is likely to have, a pathogenic infection of any kind. In some embodiments, the detection, or lack of detection of specific features, in a certain combination, may indicate the individual has, or is likely to have at least one recurrent pathogenic infection. The detection, or lack of detection of other specific features, in a certain combination, may indicate the individual has, or is likely to have, antibiotic-associated diarrhea (AAD). The detection, or lack of detection, of specific features in specific combinations may indicate the individual has a diarrheal disease, including the diseases disclosed herein. Features for a specific disease may be different between different populations of individuals. For example the detection, or lack of detection, of specific features in a sample of an adult may indicate an adult has a pathogenic infection, however the detection, or lack of detection, of the same specific features in the sample of a child may or may not indicate a child has a pathogenic infection.

In particular embodiments, the levels and/or concentrations of detected features is further compared to a known standard, wherein comparison to a known standard indicates the individual as having or not having a diarrheal disease, including a pathogenic infection, AAD, an FGID, or other diarrheal diseases disclosed herein. The levels and/or concentrations of features detected in methods of particular embodiments may be measured against known standard levels to indicate the individual has or does not have a pathogenic infection, including a potentially recurring pathogenic infection. The levels and/or concentrations of features detected in methods of particular embodiments may be measured against known standard levels to indicate the individual has or does not have AAD. The levels and/or concentrations of features detected in methods of particular embodiments may be measured against known standard levels to indicate the individual has or does not have an FGID.

In some embodiments, there may be one or more features that, when detected or not detected in a sample, are indicative of more than one diarrheal disease. In particular embodiments of the disclosure, the combination of detection, or lack of detection, of specific features in a sample from an individual indicates the individual has, or is likely to have, a specific diarrheal disease, including those disclosed herein. In some embodiments, the number of indicative features, either detected or not detected in a sample from an individual is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 1443, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 82, 183, 184, 185, 186, 187, 188, 189, 190, 191, 191, 192, 193, 194, 195, 196, 197, 198, 199, or 200 or more features encompassed herein for detecting a diarrheal disease, such as those disclosed herein. In particular embodiments, the number of indicative features, either detected or not detected in a sample from an individual is 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% features encompassed herein for detecting a diarrheal disease, such as those disclosed herein.

Particular embodiments of the disclosure concern the detection of features associated with a cellular and/or molecular response from an individual to microbiome species in the gastrointestinal tract of the individual, also known as a host response. Measuring the host response may allow for high predictively diagnosis and prognosis.

In particular embodiments, features include data collected from a sample, such as a gut, fecal, or other gastrointestinal sample. Data for identifying features as described herein may be from sequencing data, including 16S rDNA. 16S rDNA data may be used to determine the bacterial genus or species present in the sample. Data for identifying features as described herein may be from metabolomics data. Data for identifying features as described herein may be from proteomics data, which may include proteins expressed by the individual and/or proteins expressed by the microbiome located in the gastrointestinal tract of the individual.

Particular embodiments of the disclosure concern systems for measuring features from a sample, such as a gut, fecal, or other gastrointestinal sample. In particular embodiments, the system comprises one or more substrates that have molecules directly or indirectly representative of the presence of one or more features from a sample from an individual.

In particular embodiments, when the detection and/or measurement of specific features indicate an individual as having or not having a certain diarrheal disease, including a pathogenic infection, AAD, an FGID, or other diarrheal diseases disclosed herein, the individual may be administered a therapy to treat the individual. The therapy may be at least one of an antibiotic, a curative therapy, and/or a symptom relief therapy. In particular embodiments, wherein an individual is indicated to have AAD and at the time of AAD diagnosis is on an antibiotic regimen, the administration of antibiotics may be stopped, or tapered off, to reduce the cause of diarrhea, wherein the reduction of the antibiotic is a method of treatment.

Particular embodiments employ a systems-based approach to identify microbiota and host biomarkers that differentiate pathogenic cases from antibiotic-associated diarrhea (AAD) and functional gastrointestinal diseases (FGIDs). Methods, systems, and compositions encompassed in particular embodiments employ supervised learning features based on systems data generated from >2,500 fecal microbiome (16S rDNA), metaproteome, metabolome, and clinical metadata profiles from adult and pediatric cases with pathogenic infection, AAD or FGID, and control subjects without GI disease. In some aspects, pathogenic infection-classification based on fecal 16S microbiome alone data may only provide >90% diagnostic accuracy, whereas classification accuracy may improve to >99% when adding metaproteome, metabolite, and/or clinical metadata features. Importantly, these improved features confidently distinguishing pathogenic infection from potential AAD and FGID misdiagnosis. In particular embodiments, supervised learning classification of systems-based metadata offers precision diagnosis of pathogenic infection versus non-infectious enteric disease at a population scale level.

In particular embodiments, a sample is obtained from an individual suffering from symptoms of diarrhea, including acute or chronic diarrhea. The sample may be any biological sample, including any sample from the gastrointestinal tract of the individual such as a fecal sample. Levels of features, which may include nucleic acids, metabolites, proteins, clinical metadata, or other quantifiable aspects of the sample, may be measured from the sample using methods practiced by the skilled artisan. The measured levels may be analyzed, such as by applying machine learning algorithms.

In certain embodiments, the methods and systems of analyzing features utilize a so-called training set of samples from individuals with known disease states or prognoses. For example, a training set with patients known to have or not have a pathogenic infection may be used. Once established, the training data set serves as a basis, model, or template against which the features, such as features disclosed herein, of an unknown sample from an individual are compared, in order to diagnose the individual with having or not having a disease or provide a prognosis of the disease state in the individual.

Embodiments of the disclosure include methods of determining a cause of diarrhea in an individual that is suffering from diarrhea, including recurrent diarrhea. In cases wherein the diarrhea is recurrent diarrhea, a sample may be taken from an individual during a bout of diarrhea or between bouts of diarrhea. The methods of determining a cause of diarrhea comprise measuring for one or more features in one or more of Tables A-C from a gut sample from the individual, including at least a fecal sample. In some cases, the individual has two or more causes of diarrhea. Following measurement of the one or more features of one or more of Tables A-C, a treatment regimen may be determined. The treatment regimen may be effective only because the measurement of the one or more features in one or more of Tables A-C was determined. In at least some cases, were it not for the measurement of the one or more features in one or more of Tables A-C, the individual would be administered an ineffective treatment that may or may not be harmful to the individual. The treatment regimen may or may not be modulated following measurement of the one or more features in one or more of Tables A-C. In some cases, the measurement allows for confirmation of an intended treatment. In specific embodiments, the methods further comprise modulating a treatment for the individual determined to have one or more features that indicate the presence or absence of one or more conditions (or treatments therefor) that result in diarrhea. In specific embodiments, the method further comprises administering a treatment or reducing a treatment to the individual when the individual is determined to have one or more features that indicate the presence or absence of one or more diarrheal-associated diseases. In specific embodiments, the individual having one or more particular features in one or more of Tables A-C is determined to have an infection of one or more pathogens. In specific embodiments, the individual having one or more particular features is determined to have antibiotic-associated diarrhea and, in at least some cases, the antibiotic is halted or reduced in dosage following such determination.

Any method encompassed herein may utilize measuring of one or more features disclosed herein. The measuring in at least some cases identifies the presence or absence of one or more features encompassed in the disclosure herein. In some cases, the measuring identifies a level of one or more features encompassed in the disclosure herein, including a level that is compared to a threshold or known standard. Any suitable control, threshold or known standard may be utilized, but in specific embodiments the threshold or known standard is a reference from age-matched and/or sex-matched individuals who do not have diarrhea or do not have recurrent diarrhea.

Any mammalian individual susceptible to toxins of a pathogen may be subject to methods of the disclosure. The individual may be of any gender or age, including an adult, child, or infant. However, in specific embodiments, the individual is of a sufficient age to be susceptible to toxins of a pathogen, including at least or at least about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months of age. The individual may or may not have recurrent diarrhea. The individual may or may not be suspected of having misdiagnosis of a cause for any diarrhea, including recurrent diarrhea. The individual may be subject to methods of the disclosure to avoid having a misdiagnosis of a cause for any diarrhea, including recurrent diarrhea.

Methods of the disclosure include methods of treating an individual having diarrhea (recurrent or not) comprising measuring for one or more features encompassed in one or more of Tables A-C from a gut sample (including fecal sample) from the individual; and either (1) reducing the administration of one or more antibiotics to the individual when the individual has presence or absence or a certain level of one or more feature(s) encompassed in one or more of Tables A-C, for example said features being indicative of antibiotic associated diarrhea; or (2) administering one or more antibiotics and/or antimicrobials to the individual when the individual has presence or absence or a certain level of one or more feature(s) indicative of a pathogen infection, for example said features being indicative of a pathogen infection.

Methods of the disclosure include methods of treating an individual having diarrhea (recurrent or not) comprising measuring for one or more features encompassed in one or more of Tables A-C from a gut sample (including fecal sample) from the individual; and either (1) reducing the administration of one or more antibiotics for an individual determined to have the presence or absence or a certain level of one or more feature(s) encompassed in one or more of Tables A-C, for example said one or more features being indicative of antibiotic associated diarrhea; or (2) administering one or more antibiotics to an individual determined to have the presence or absence or a certain level of one or more feature(s) encompassed in one or more of Tables A-C, for example said features being indicative of a pathogen infection.

Any antibiotics and/or antimicrobials to be provided to the individual when appropriate or to be avoided for the individual when appropriate may comprise at least one of the antibiotics and/or antimicrobials selected from the group consisting of a small molecule antibiotic, an antibiotic derived from a natural product, a microbial composition, an antibody or therapeutic suitable for neutralizing pathogenic infections, and a combination thereof.

Embodiments of the disclosure include methods of measuring one or more features encompassed herein in a fecal or gut sample from an individual that has diarrhea, that has recurrent diarrhea, and/or that is suspected of having a misdiagnosis of a diarrheal cause, comprising the steps of two or more of the following: analyzing one or more nucleic acids in the sample; analyzing one or more metabolites in the sample; and analyzing one or more proteins in the sample. In specific embodiments, the analyzing includes analyzing for the presence and/or level of one or more features encompassed in one or more of Tables A-C. In cases wherein the nucleic acid from a sample is analyzed, the nucleic acid may be analyzed by sequencing, polymerase chain reaction, isothermal amplification, bioinformatics, or a combination thereof. The nucleic acid may be of any kind that is indicative of presence of a pathogen, such as 16S ribosomal RNA. Any nucleic acid analysis may or may not include whole genome sequencing, yet in specific cases it does not include whole genome sequencing. In cases wherein metabolites from a sample are analyzed, the analysis may be by mass spectrometry, ELISA, chromatography, or a combination thereof. In cases wherein proteins are analyzed from a sample, the proteins may be analyzed by mass spectrometry, ELISA, chromatography, Western blotting, immunoprecipitation, immunoelectrophoresis, or a combination thereof.

Embodiments of the disclosure include methods to measure a host response to a microbial infection in an individual, said individual that has diarrhea, that has recurrent diarrhea, and/or that is suspected of having a misdiagnosis of a diarrheal cause, comprising the steps of analyzing one or more nucleic acids in a fecal or gut sample from the individual; analyzing metabolites in the sample; and/or analyzing proteins in the sample. In such methods, the microbial infection may be of any kind that causes diarrhea in a host, but in specific embodiments the infection is any species of a pathogen that can cause diarrhea in a host. In such methods, the one or more features are encompassed in one or more of Tables A-C.

In particular embodiments of the disclosure, one identifies whether or not an individual is high risk, moderate risk or low risk of having pathogenic infection. Such embodiments include the ability to predict an outcome for the individual. Any analysis for any method herein may occur at the time that an individual has diarrhea, at the time or after that an individual has a second or subsequent bout of diarrhea, or as part of routine screening for general health purposes.

In specific embodiments, an individual is not subject to methods of disclosure unless they have had antibiotics and/or antimicrobials, given that generally healthy adults have a low risk of pathogenic infection unless they take antibiotics. Therefore, in specific embodiments a sample from an individual is measured for one or more feature(s) as encompassed herein before antibiotics and/or antimicrobials are administered, while antibiotics and/or antimicrobials are being administered, and/or after antibiotics and/or antimicrobials have been administered. The course of antibiotics or any antimicrobial treatment including chemotherapy may be a first exposure for the individual, although in some cases it is a second or subsequent exposure to antibiotics.

In particular methods of the disclosure, individuals with or at risk for pathogenic infection are able to be distinguished from individuals with our at risk for irritable bowel syndrome (IBS). In some cases, an individual with a first or subsequent bout of diarrhea is subjected to methods of the disclosure in which case one or more particular features identify an individual with or at risk for pathogenic infection or not as having or at risk for pathogenic infection. In some cases, pathogenic infection may be ruled out as a cause or risk for the individual and the individual is then determined whether or not they have IBS, whether or not that IBS determination utilizes information from feature(s) of the disclosure.

In pediatric individuals, some are of an early enough age that they are not yet susceptible to toxins from one or more pathogens, and yet they may be subjected to methods of the disclosure to determine their risk of pathogenic infection once they become old enough to be susceptible to the toxins. In some cases, the individual is not subjected to methods of the disclosure until they are suspected or shown to be susceptible to the toxins, for example suspected because they reach a certain age. Any of such screening methods may be performed as routine health care for the pediatric individual.

Embodiments of the disclosure allow for distinguishing whether or not features for an individual are suitable for indicating the presence or risk for pathogenic infection. In specific cases, the form of features that are analyzed needs to be indicative of the presence of live bacteria capable of producing toxins that cause diarrhea as opposed to dead bacteria that cannot. Therefore, in at least some cases one or more features that are used are not nucleic acid in form because nucleic acids may originate from dead bacteria. In specific cases, one or more non-nucleic acid features that represent metabolic activity are utilized to identify the presence of live bacteria that may be causing diarrhea, such as metabolites that may be small molecules and/or proteins.

Embodiments of the disclosure encompass methods wherein outcome of a therapy for pathogenic infection patients, including recurrent pathogenic infection, is predictable or determined based on the measurement of one or more features from one or more of Tables A-C. The therapy may be of any kind, including at least FMT, antibiotics, therapeutics, contact isolation, or a combination thereof.

Methods and compositions of the disclosure can distinguish an individual that has irritable bowel syndrome (IBS) versus an individual that has a pathogenic infection. In specific cases, an individual having certain one or more features from one or more of Tables A-C is determined to have IBS instead of a pathogenic infection, and in specific embodiments following this determination the individual is accurately treated for IBS instead of a pathogenic infection. In other cases, an individual having certain one or more features from one or more of Tables A-C is determined to have a pathogenic infection instead of IBS, and in specific embodiments following this determination the individual is accurately treated for a pathogenic infection instead of IBS.

Methods and compositions of the disclosure can distinguish an individual that has antibiotic-associated diarrhea versus an individual that has a pathogenic infection. In specific cases, an individual having certain one or more features from one or more of Tables A-C is determined to have antibiotic-associated diarrhea instead of a pathogenic infection, and in specific embodiments following this determination the individual is accurately treated for antibiotic-associated diarrhea instead of a pathogenic infection. In other cases, an individual having certain one or more features from one or more of Tables A-C is determined to have a pathogenic infection instead of antibiotic-associated diarrhea, and in specific embodiments following this determination the individual is accurately treated for a pathogenic infection instead of antibiotic-associated diarrhea.

IV. Features and Compositions

Embodiments of the disclosure include the one or more features encompassed in one or more of Tables A-C. Such features may be embodied as a grouping of indicators for having a pathogenic infection, for not having a pathogenic infection, for being at risk for having a pathogenic infection, or not for being at risk for having a pathogenic infection. In specific cases, such features may be embodied as a grouping of indicators for having CDI, for not having CDI, for being at risk for CDI, or not for being at risk for CDI. The features may be exemplified in the forms of nucleic acid, protein (or peptide(s)), or small molecules (such as with metabolites). In some cases, a feature may be utilized in two types or three or more types of forms (such as nucleic acid, metabolite, lipid, and protein). In particular cases, the features may be represented in any form on a substrate for measuring, such as an assay substrate. Specific embodiments comprise microassay susbstrates for measuring one or more features encompassed in one or more of Tables A-C.

Any feature for determining diagnosis related to whether or not an individual has a pathogenic infection (including at least CDI) may be an indicator from a microbe in the individual or from the host individual. In some cases, a grouping of features are indicators whether or not an individual has diarrhea from pathogenic infection (including at least CDI) or from another cause, and this grouping may include one or more features from the host individual (for example, metabolites from host cells) and/or may include one or more features from one or more microbes within the host individual, including whether or not those one or more microbes are pathogenic to the host themselves.

In specific embodiments, the determination whether or not an individual has a pathogenic infection (including at least CDI) or has diarrhea from a non-CDI cause (including another pathogen) includes analysis of any one or more features from one or more of Tables A-C. In specific cases, the features is exactly or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 1443, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 82, 183, 184, 185, 186, 187, 188, 189, 190, 191, 191, 192, 193, 194, 195, 196, 197, 198, 199, or 200 or more features encompassed herein. The feature may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of the features encompassed herein.

In specific embodiments, the feature(s) indicative of whether or not an individual has a pathogenic infection or whether or not the individual is at risk for pathogenic infection comprises one or more features from Table A, one or more features from Table B, and/or one or more features from Table C.

In particular embodiments, the feature(s) indicative of whether or not an individual has pathogenic infection or whether or not the individual is at risk for pathogenic infection may utilize different features in different forms. For example, a determination of outcome from the methods may utilize nucleic acid analysis for one or more features, protein analysis for one or more features, and/or small molecular analysis for one or more features. In specific embodiments, however, the features are measured as the form, such as all of the features for the methods being nucleic acid, all of the features being proteins, and/or all of the features being small molecules.

Features encompassed in the disclosure allow discrimination of pathogenic infection-related embodiments versus non-pathogenic infection-related embodiments. Although the features(s) may be analyzed qualitatively as measurement for whether or not an individual has pathogenic infection or is at risk for pathogenic infection, in particular embodiments the features(s) are analyzed quantitatively. Such quantitative analysis may be with respect to a control, including a control level of the feature in question from a population of individuals that lack pathogenic infection, are not at risk for pathogenic infection, or that do not have diarrhea, including recurrent diarrhea.

One or more features may or may not be enriched in a sample with respect to a respective control and/or one or more features may be deficient in a sample with respect to a respective control. Certain one or more features may have a magnitude of an increase or decrease with respect to a control that is indicative of having or being at risk for pathogenic infection, or not. In specific cases, a feature is a certain fold level increase or decrease over a control level, dependent upon the feature. For example, an individual may have a 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 50-fold or more increase in level of a certain feature over a control level. An individual may have a 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 50-fold or more decrease in level of a certain feature over a control level, in some cases.

Table A lists examples of features that may be assayed in the form of nucleic acid, such as 16S rRNA gene amplicon sequencing. Table A delineates specific features and the magnitude and directional change of level in the right column. For features that show an arrow pointing up, relative abundance of these predictive features are increased in 16S rRNA gene level in control samples as compared to individuals that have pathogenic infection or are at risk for pathogenic infection. For features that show an arrow pointing down, these features are decreased in 16S rRNA gene level in control samples as compared to individuals that have pathogenic infection or are at risk for pathogenic infection.

Therefore, compared to a control, an individual that has pathogenic infection or that is at risk for pathogenic infection would have decreased levels of all features with arrows pointing up and the same individual would have increased levels of predictive features with arrows pointing down.

As one example in the first row of Table A, Bacteroides is increased in control levels by a 2.1 fold change when compared to a sample from an individual with pathogenic infection or at risk thereof. Therefore, if a sample of an individual suspected of having or being at risk for pathogenic infection had a level of Bacteroides that was about 2.1-fold or greater fold change decreased with respect to a control, then that individual has pathogenic infection or is at risk for pathogenic infection. As another example, if there is a 2.13 fold change, this means that 213% increase relative level in controls versus pathogenic infection.

Such denotation of arrows and fold change also applies to Tables B and C.

In specific embodiments, Table A provides a list of exemplary features for determination of whether or not an individual has pathogenic infection or is at risk for pathogenic infection.

TABLE A
Examples of 16S rRNA Features

| | | Control vs. |
|---|---|---|
| Rank # | | CDI or other |
| (ANOVA F- | | pathogen |
| Values) | Feature description | Fold change |
| 1 | Bacteria; Bacteroidetes; Bacteroidia; Bacteroidales; | ↑ 2.13017194 |
| | Bacteroidaceae; Bacteroides | |
| 2 | Bacteria; Firmicutes; Clostridia; Clostridiales; | ↑ 14.99789803 |
| | Lachnospiraceae; [Eubacterium] rectale group | |
| 3 | Bacteria; Firmicutes; Clostridia; Clostridiales; | ↑ 8.009914311 |
| | Ruminococcaceae; Ruminococcus | |
| 4 | Bacteria; Firmicutes; Clostridia; Clostridiales; | ↑ 4.127913445 |
| | Ruminococcaceae; Faecalibacterium | |
| 5 | Bacteria; Firmicutes; Bacilli; Lactobacillales; | ↓ 0.012322411 |
| | Enterococcaceae; Enterococcus | |
| 6 | Bacteria; Proteobacteria; Gammaproteobacteria; | ↓ 0.0793284 |
| | Enterobacteriales; Enterobacteriaceae; Other | |
| 7 | Bacteria; Firmicutes; Clostridia; Clostridiales; | ↑ 3.915975647 |
| | Lachnospiraceae; Roseburia | |
| 8 | Bacteria; Firmicutes; Clostridia; Clostridiales; | ↑ 8.166342647 |
| | Lachnospiraceae; Coprococcus | |
| 9 | Bacteria; Firmicutes; Clostridia; Clostridiales; | ↑ 6.301045916 |
| | Lachnospiraceae; Dorea | |
| 10 | Bacteria; Firmicutes; Clostridia; Clostridiales; Lachnospirac | ↓ 0.097496417 |
| | eae; Lachnoclostridium | |
| 11 | Bacteria; Firmicutes; Clostridia; Clostridiales; | ↓ 0.090464494 |
| | Lachnospiraceae; Clostridium X1Va | |
| 12 | Bacteria; Firmicutes; Erysipelotrichia; Erysipelotrichales; | ↓ 0.135798332 |
| | Erysipelotrichaceae; Erysipelatoclostridium | |
| 13 | Bacteria; Bacteroidetes; B acteroidia; B acteroidales; | ↑ 2.62180625 |
| | Rikenellaceae; Alistipes | |
| 14 | Bacteria; Firmicutes; Clostridia; Clostridiales; | ↑ 5.425625745 |
| | Lachnospiraceae; Fusicatenibacter | |
| 15 | Bacteria; Bacteroidetes; Bacteroidia; | ↑ 4.822822775 |
| | Bacteroidales; Porphyromonadaceae; Odoribacter | |
| 16 | Bacteria; Firmicutes; Bacilli; Lactobacillales; | ↓ 0.106678394 |
| | Lactobacillaceae; Lactobacillus | |
| 17 | Bacteria; Firmicutes; Clostridia; Clostridiales; | ↑ 2.982836857 |
| | Lachnospiraceae;Anaerostipes | |
| 18 | Bacteria;Actinobacteria;Coriobacteriia;Coriobacteriales; | ↑ 5.551036189 |
| | Coriobacteriaceae; Collinsella | |
| 19 | Bacteria; Firmicutes; Clostridia; Clostridiales; | ↓ 0.001872429 |
| | Peptostreptococcaceae; Clostridioides | |
| 20 | Bacteria; Firmicutes; Clostridia; Clostridiales; | ↑ 2.426307825 |
| | Ruminococcaceae; Other | |
| 21 | Bacteria; Firmicutes; Clostridia; Clostridiales; | ↑ 2.340306136 |
| | Lachnospiraceae; Lachnospiracea_incertae_sedis | |
| 22 | Bacteria; Firmicutes; Clostridia; Clostridiales; Other; Other | ↑ 2.247997661 |
| 23 | Bacteria; Proteobacteria; Gammaproteobacteria; | ↓ 0.060177638 |
| | Enterobacterales; Enterobacteriaceae; Klebsiella | |
| 24 | Bacteria; Firmicutes; Clostridia; Clostridiales; | ↑ 6.604612787 |
| | Agathobaculum; Agathobaculum butyriciproducens | |
| 25 | Bacteria; Proteobacteria; Gammaproteobacteria; Other; | ↓ 0.142279533 |
| | Other; Other | |
| 26 | Bacteria; Firmicutes; Negativicutes; Veillonellales; | ↓ 0.035621822 |
| | Veillonellaceae; Veillonella | |
| 27 | Bacteria; Firmicutes; Negativicutes; Acidaminococcales; | ↑ 2.783965319 |
| | Acidaminococcaceae; Phascolarctobacterium | |
| 28 | Bacteria; Actinobacteria; Coriobacteriia; Eggerthellales; | ↑ 7.25114612 |
| | Eggerthellaceae; Adlercreutzia | |
| 29 | Bacteria; Firmicutes; Clostridia; Clostridiales; | ↓ 0.181659448 |
| | Clostridiaceae; Clostridium | |
| 30 | Bacteria; Actinobacteria; Coriobacteriia; Eggerthellales; | ↓ 0.219042004 |
| | Eggerthellaceae; Eggerthella | |
| 31 | Bacteria; Proteobacteria; Betaproteobacteria; | ↑ 3.727917577 |
| | Burkholderiales; Sutterellaceae; Parasutterella | |
| 32 | Bacteria; Bacteroidetes; Bacteroidia; Bacteroidales; | ↑ 5.083698415 |
| | Porphyromonadaceae; Barnesiella | |
| 33 | Bacteria; Firmicutes; Clostridia; Clostridiales; | ↑ 2.509692973 |
| | Eubacteriaceae; Eubacterium | |
| 34 | Bacteria; Bacteroidetes; Bacteroidia; Bacteroidales; | ↑ 2.784113311 |
| | Odoribacteraceae; Odoribacter | |
| 35 | Bacteria; Firmicutes; Clostridia; Clostridiales; | ↑ 3.929516525 |
| | Ruminococcaceae; Clostridium IV | |
| 36 | Bacteria; Firmicutes; Negativicutes; Selenomonadales; | ↑ 4.920265286 |
| | Acidaminococcaceae; Phascolarctobacterium | |
| 37 | Bacteria; Actinobacteria; Actinobacteria; Coriobacteriales; | ↓ 0.102896013 |
| | Coriobacteriaceae; Eggerthella | |
| 38 | Bacteria; Firmicutes; Clostridia; Clostridiales; | ↑ 3.120301808 |
| | Ruminococcaceae; Gemmiger | |
| 39 | Bacteria; Firmicutes; Negativicutes; Selenomonadales; | ↓ 0.027411325 |
| | Veillonellaceae; Veillonella | |
| 40 | Bacteria; Firmicutes; Clostridia; Clostridiales; | ↑ 1.631745536 |
| | Lachnospiraceae; Other | |
| 41 | Bacteria; Firmicutes; Bacilli; Lactobacillales; Other; Other | ↓ 0.001895411 |
| 42 | Bacteria; Firmicutes; Bacilli; Lactobacillales; | ↓ 0.342142181 |
| | Streptococcaceae; Streptococcus | |
| 43 | Bacteria; Firmicutes; Negativicutes; Selenomonadales; | ↑ 5.366776645 |
| | Veillonellaceae; Dialister | |
| 44 | Bacteria; Proteobacteria; Gammaproteobacteria; | ↓ 0.299446869 |
| | Enterobacterales; Enterobacteriaceae; Escherichia | |
| 45 | Bacteria; Firmicutes; Clostridia; Clostridiales; Not | ↑ 17.52578836 |
| | Available; Colidextribacter | |
| 46 | Bacteria; Proteobacteria; Betaproteobacteria; | ↑ 11.04826253 |
| | Burkholderiales; Oxalobacteraceae; Oxalobacter | |
| 47 | Bacteria; Bacteroidetes; Bacteroidia; Bacteroidales; | ↑ 3.816100602 |
| | Prevotellaceae; Prevotella | |
| 48 | Bacteria; Firmicutes; Erysipelotrichia; Erysipelotrichales; | ↓ 0.163190685 |
| | Erysipelotrichaceae; Clostridium XVIII | |
| 49 | Bacteria; Firmicutes; Bacilli; Lactobacillales; | ↓ 0.001286643 |
| | Enterococcaceae; Other | |
| 50 | Bacteria; Firmicutes; Clostridia; Clostridiales; | ↑ 8.534861125 |
| | Ruminococcaceae; Agathobaculum | |
| 51 | Bacteria; Actinobacteria; Actinobacteria; Actinomycetales; | ↓ 0.181422182 |
| | Actinomycetaceae; Actinomyces | |
| 52 | Bacteria; Fusobacteria; Fusobacteriia; Fusobacteriales; | ↓ 0.056291487 |
| | Fusobacteriaceae; Fusobacterium | |

TABLE B
Examples of Metaproteome Features from a Human Host and from
a Microbiome of the Human Host

| | | | | Control vs CDI |
|---|---|---|---|---|
| Rank # by | | | | or other |
| ANOVA | | | | pathogen |
| F-Values | Feature ID | Feature_taxonomy | Feature_function | Fold change |
| 1 | P01833 | Human | Polymeric | ↓ 0.20704087 |
| | | | immunoglobulin | |
| | | | receptor | |
| 2 | E7EQB2 | Human | Lactotransferrin | ↓ 0.052901269 |
| | | | (Fragment) | |
| 3 | P05164 | Human | Myeloperoxidase | ↓ 0.042833644 |
| 4 | P05109 | Human | Protein S100-A8 | ↓ 0.064770134 |
| 5 | MH0355_GL00381 | d_Bacteria; p_Firmicutes; | pyruvate- | ↑ 44.32861379 |
| | 20 | c_Clostridia; o_Clostridiales; | ferredoxin/flavodoxin | |
| | | f_Lachnospiraceae; unclassified; | oxidoreductase | |
| | | unclassified | | |
| 6 | MH0131_GL01354 | d_Bacteria; p_Firmicutes; | raffinose/stachyose/ | ↑ 1000.00 |
| | 07 | c_Clostridia; o_Clostridiales; | melibiose transport | |
| | | f_Ruminococcaceae; | system substrate- | |
| | | g_Faecalibacterium; | binding protein | |
| | | s_Faecalibacterium prausnitzii | | |
| 7 | P01619 | Human | Immunoglobulin | ↓ 0.051582427 |
| | | | kappa variable 3-20 | |
| 8 | P11678 | Human | Eosinophil peroxidase | ↓ 0.105465287 |
| 9 | P08246 | Human | Neutrophil elastase | ↓ 0.033685382 |
| 10 | V1.FI06_GL00601 | d_Bacteria; p_Firmicutes; | acetyl-CoA C- | ↑ 151.2140889 |
| | 06 | c_Clostridia; o_Clostridiales; | acetyltransferase | |
| | | unclassified; unclassified; | | |
| | | unclassified | | |
| 11 | MH0002_GL00546 | d_Bacteria; p_Firmicutes; | ketol-acid | ↑ 22.52923559 |
| | 36 | c_Clostridia; o_Clostridiales; | reductoisomerase | |
| | | unclassified; unclassified; | | |
| | | unclassified | | |
| 12 | Q9BYE9 | Human | Cadherin-related | ↑ 7.47304609 |
| | | | family member 2 | |
| 13 | O2.UC15- | d_Bacteria; p_Firmicutes; | ethanolamine | ↑ 82.11005268 |
| | 0_GL0062928 | c_Clostridia; o_Clostridiales; | utilization protein | |
| | | f_Eubacteriaceae; unclassified; | EutM | |
| | | unclassified | | |
| 14 | A0A1B0GUU9 | Human | Immunoglobulin | ↓ 0.070358532 |
| | | | heavy constant mu | |
| | | | (Fragment) | |
| 15 | P24158 | Human | Myeloblastin | ↓ 0.076600946 |
| 16 | P08311 | Human | Cathepsin G | ↓ 0.029757122 |
| 17 | Q9UGM3 | Human | Deleted in malignant | ↓ 0.277315579 |
| | | | brain tumors 1 | |
| | | | protein | |
| 18 | MH0198_GL00799 | d_Bacteria; p_Firmicutes; | L-fucose/D-arabinose | ↑ 10.27608806 |
| | 53 | c_Clostridia; o_Clostridiales; | isomerase | |
| | | unclassified; unclassified; | | |
| | | unclassified | | |
| 19 | P06702 | Human | Protein S100-A9 | ↓ 0.06011116 |
| 20 | 763840445- | d_Bacteria;p_Finnicutes; | lactose/L-arabinose | ↑ 22.24817166 |
| | stool2_revised_{—} | c_Clostridia; o_Clostridiales; | transport system | |
| | scaffold50645_1_{—} | unclassified; unclassified; | substrate-binding | |
| | gene95245 | unclassified | protein | |
| 21 | O2.UC57- | d_Bacteria; p_Firmicutes; | acetyl-CoA C- | ↑ 20.10400416 |
| | 2_GL0097093 | c_Clostridia; o_Clostridiales; | acetyltransferase | |
| | | f_Eubactcriaceae; | | |
| | | g_Eubacterium; unclassified | | |
| 22 | MH0441_GL00535 | d_Bacteria; p_Firmicutes; | formate C- | ↑ 73.90616117 |
| | 60 | c_Clostridia; o_Clostridiales; | acetyltransferase | |
| | | f_Eubacleriaceae; | | |
| | | g_Eubacterium; unclassified | | |
| 23 | Q8WWA0 | Human | Intelectic-1 | ↓ 0.299627479 |
| 24 | P20160 | Human | Azurocidin | ↓ 0.017131544 |
| 25 | MH0002_GL00227 | d_Bacteria; p_Firmicutes; | acetyl-CoA C- | ↑ 1000.00 |
| | 47 | c_Clostridia; o_Clostridiales; | acetyltransferase | |
| | | f_Lachnospiraceae; | | |
| | | g_Roseburia; unclassified | | |
| 26 | MH0233_GL00353 | d_Bacteria; p_Firmicutes; | ribose transport | ↑ 76.55358737 |
| | 17 | c_Clostridia; o_Clostridiales; | system substrate- | |
| | | f_Ruminococcaceae; | binding protein | |
| | | g_Ruminococcus; unclassified | | |
| 27 | A8K7I4 | Human | Calcium-activated | ↓ 0.421181613 |
| | | | chloride channol | |
| | | | regulator 1 | |
| 28 | A0A286YEY1 | Human | Immunoglobulin | ↓ 0.503979394 |
| | | | heavy constant alpha | |
| | | | 1 (Fragment) | |
| 29 | MH0264_GL00810 | d_Bacteria; p_Firmicutes; | multiple sugar | ↑ 1000.00 |
| | 16 | c_Clostridia; o_Clostridiales; | transport system | |
| | | f_Lachnospiraceae; | ATP-binding protein | |
| | | g_Roseburia; unclassified | | |
| 30 | MH-0161_GL00393 | d_Bacteria; p_Firmicutes; | L-fucose/D-arabinose | ↑ 14.92921126 |
| | 59 | c_Clostridia; o_Clostridiales; | isomerase | |
| | | unclassified; unclassified; | | |
| | | unclassified | | |
| 31 | P01834 | Human | Immunoglobulin | ↓ 0.389073968 |
| | | | kappa constant | |
| 32 | Q9H3R2 | Human | Mucin-13 | ↓ 0.093496438 |
| 33 | D6RD17 | Human | Immunoglobulin J | ↓ 0.228872292 |
| | | | chain (Fragment) | |
| 34 | P01024 | Human | Complement C3 | 0 |
| 35 | MH0110_GL00252 | d_Bacteria; p_Firmicutes; | pyruvate, | ↑ 10.02875745 |
| | 97 | c_Clostridia; o_Clostridiales; | orthophosphate | |
| | | f_Ruminococcaceae; | dikinase | |
| | | g_Ruminococcus; unclassified | | |
| 36 | 160502038- | d_Bacteria; p_Firmicutes; | pyruvate- | ↑ 10.08302206 |
| | stool1_revised_{—} | c_Clostridia; o_Clostridiales; | ferredoxin/flavodoxin | |
| | scaffold27245_1_{—} | f_Ruminococcaceae; | oxidoreductase | |
| | gene99702 | g_Ruminococcus; unclassified | | |
| 37 | DLM014_GL00049 | d_Bacteria; p_Firmicutes; | raffinose/stachyose/ | ↑ 1000.00 |
| | 97 | c_Clostridia; o_Clostridiales; | melibiose transport | |
| | | f_Ruminococcaceae; | system substrate- | |
| | | g_Faecalibacterium; | binding protein | |
| | | s_Faecalibacterium prausnitzii | | |
| 38 | P02814 | Human | Submaxillary gland | ↑ 10.48327841 |
| | | | androgen-regulated | |
| | | | protein 3B | |
| 39 | P01023 | Human | Alpha-2- | ↓ 0.082863448 |
| | | | macroglobulin | |
| 40 | 764-62976- | d_Bacteria; p_Firmicutes; | phosphoenolpyruvate | ↑ 1000.00 |
| | stool1_revised_{—} | c_Clostridia; o_Clostridiales; | carboxykinase (ATP) | |
| | C1170782_1_{—} | unclassified; unclassified; | | |
| | gene8032 | unclassified | | |
| 41 | 159551223- | d_Bacteria; p_Firmicutes; | phosphoenolpyruvate | ↑ 32.63930228 |
| | stool2_revised_{—} | c_Clostridia; o_Clostridiales; | carboxykinase (ATP) | |
| | scaffold41270_1_{—} | f_Lachnospiraceae; unclassified; | | |
| | gene16557 | unclassified | | |
| 42 | MH0014_GL00259 | d_Bacteria; p_Proteobacteria; | murein lipoprotein | 0 |
| | 05 | c_Gammaproteobacteria; | | |
| | | o_Enterobacterales; | | |
| | | f_Enterobacteriaceae; | | |
| | | g_Escherichia; s_Escherichia coli | | |
| 43 | A0A0C4DGB6 | Human | Serum albumin | 0 |
| 44 | MH0044_GL00665 | d_Bacteria; p_Firmicutes; | raffinose/stachyose/ | ↑ 10.48838135 |
| | 39 | c_Clostridia; o_Clostridiales; | melibiose transport | |
| | | f_Ruminococcaceae; | system substrate- | |
| | | g_Gemmiger; s_Gemmiger | binding protein | |
| | | formicilis | | |
| 45 | 159753524- | d_Bacteria; p_Firmicutes; | large subunit | ↑ 8.138378882 |
| | stool1_revised_{—} | c_Clostridia; o_Clostridiales; | ribosomal protein L4 | |
| | C1069796_1_{—} | f_Lachnospiraceae; | | |
| | gene63983 | unclassified; unclassified | | |
| 46 | DLM020_GL00252 | d_Bacteria; p_Firmicutes; | raffinose/stachyose/ | ↑ 1000.00 |
| | 26 | c_Clostridia; o_Clostridiales; | melibiose transport | |
| | | f_Ruminococcaceae; | system substrate- | |
| | | g_Faecalibacterium; | binding protein | |
| | | s_Faecalibacterium prausnitzii | | |
| 47 | 764062976- | d_Bacteria; p_Firmicutes; | large subunit | ↑ 20.12912405 |
| | stool1_revised_{—} | c_Clostridia; o_Clostridiales; | ribosomal protein L4 | |
| | scaffold37946_1_{—} | f_Lachnospiraceae; | | |
| | gene15553 | g_unknown; s_Lachnospiraceae | | |
| | | bacterium V9D3004 | | |
| 48 | 765013792- | d_Bacteria; p_Firmicutes; | simple sugar transport | ↑ 20.1879946 |
| | stool1_revised_{—} | c_Clostridia; o_Clostridiales; | system substrate- | |
| | C383107_1_{—} | f_Ruminococcaceae; | binding protein | |
| | gene19778 | g_Ruminococcus; unclassified | | |
| 49 | MH0088_GL00182 | d_Bacteria; p_Firmicutes; | L-fucose/D-arabinose | ↑ 40.02867755 |
| | 97 | c_Clostridia; o_Clostridiales; | isomerase | |
| | | unclassified; unclassified; | | |
| | | unclassified | | |
| 50 | MH0087_GL00085 | d_Bacteria; p_Firmicutes; | anaerobic carbon- | ↑ 10.85822534 |
| | 88 | c_Clostridia; o_Clostridiales; | monoxide | |
| | | f_Ruminococcaceae; | dehydrogenase | |
| | | g_Ruminococcus; unclassified | catalytic subunit | |
| 51 | MH0006_GL00212 | d_Bacteria; p_Firmicutes; | acetyl-CoA | ↑ 38.12838404 |
| | 87 | c_Clostridia; o_Clostridiales; | decarbonylase/ | |
| | | unclassified; unclassified; | synthase, CODH/ACS | |
| | | unclassified | complex subunit | |
| | | | gamma | |
| 52 | 158802708- | d_Bacteria; p_Firmicutes; | acetyl-CoA C- | ↑ 1000.00 |
| | stool1_revised_{—} | c_Clostridia; o_Clostridiales; | acetyltransferase | |
| | C973589_1_{—} | f_Lachnospiraceae; | | |
| | gene123870 | g_Roseburia; unclassified | | |
| 53 | 657314.CK5_22910 | d_Bacteria; p_Firmicutes; | aldehyde | ↑ 22.99089409 |
| | | c_Clostridia; o_Clostridiales; | oxidoreductase | |
| | | unclassified; unclassified; | | |
| | | unclassified | | |
| 54 | MH0060_GL00464 | d_Bacteria; p_Firmicutes; | glutamate | ↑ 56.76560879 |
| | 90 | c_Clostridia; o_Clostridiales; | dehydrogenase | |
| | | f_Ruminococcaceae; | (NADP+) | |
| | | g_Faecalibacterium; | | |
| | | s_Faecalibacterium prausnitzii | | |
| 55 | P59665 | Human | Neutrophil defensin 1 | ↓ 0.159655159 |
| 56 | P13688 | Human | Carcinoembryonic | ↓ 0.090315204 |
| | | | antigen-related cell | |
| | | | adhesion molecule 1 | |
| 57 | P12724 | Human | Eosinophil cationic | ↓ 0.022244618 |
| | | | protein | |
| 58 | 160218816- | d_Bacteria; p_Firmicutes; | glutamate | ↑ 1000.00 |
| | stool1_revised_{—} | c_Clostridia; o_Clostridiales; | dehydrogenase | |
| | scaffold41950_1_{—} | unclassified; unclassified; | (NADP+) | |
| | gene92719 | unclassified | | |
| 59 | P15144 | Human | Aminopeptidase N | ↓ 0.219604453 |
| 60 | P13727 | Human | Bone marrow | 0 |
| | | | proteoglycan | |
| 61 | 765560005- | d_Bacteria; p_Firmicutes; | PTS system, N- | ↑ 1000.00 |
| | stool1_revised_{—} | c_Clostridia; o_Clostridiales; | acetylglucosamine- | |
| | scaffold3161_6_{—} | f_Ruminococcaceae; | specific IIB | |
| | gene28967 | g_Faecalibacterium; | component | |
| | | s_Faecalibacterium prausnitzii | | |
| 62 | A0A024R0K5 | Human | Carcinoembryonic | ↓ 0.284614173 |
| | | | antigen-related cell | |
| | | | adhesion molecule 5, | |
| | | | isoform CRA_a | |
| 63 | MH0203_GL01849 | d_Bacteria; p_Firmicutes; | L-fucose/D-arabinose | ↑ 1000.00 |
| | 45 | c_Clostridia; o_Clostridiales; | isomerase | |
| | | unclassified; unclassified; | | |
| | | unclassified | | |
| 64 | P80188 | Human | Neutrophil gelatinase- | ↓ 0.033197651 |
| | | | associated lipocalin | |
| 65 | P05451 | Human | Lithostathine-1-alpha | ↓ 0.084222355 |
| 66 | 764062976- | d_Bacteria; p_Firmicutes; | glyceraldehyde 3- | ↑ 19.84978295 |
| | stool1_revised_{—} | c_Clostridia; o_Clostridiales; | phosphate | |
| | scaffold43051_1_{—} | unclassified; unclassified; | dehydrogenase | |
| | gene64550 | unclassified | | |
| 67 | 764062976- | d_Bacteria; p_Firmicutes; | 3-hydroxybutyryl- | ↑ 17.0174367 |
| | stool2_revised_{—} | c_Clostridia; o_Clostridiales; | CoA dehydrogenase | |
| | C1008473_1_{—} | unclassified; unclassified; | | |
| | gene94482 | unclassified | | |
| 68 | 764447348- | d_Bacteria; p_Firmicutes; | 3-hydroxybutyryl- | ↑ 9.459028726 |
| | stool1_revised_{—} | c_Clostridia; o_Clostridiales; | CoA dehydrogenase | |
| | C414751_1_{—} | f_Ruminococcaceae; | | |
| | gene72200 | g_Faecalibacterium; | | |
| | | s_Faecalibacterium prausnitzii | | |
| 69 | MH0127_GL00277 | d_Bacteria; p_Firmicutes; | pyruvate, | ↑ 1000.00 |
| | 09 | c_Clostridia; o_Clostridiales; | orthophosphate | |
| | | f_Lachnospiraceae; | dikinase | |
| | | unclassified; unclassified | | |
| 70 | MH0088_GL00800 | d_Bacteria; p_Firmicutes; | peptide/nickel | ↑ 43.58224868 |
| | 36 | c_Clostridia; o_Clostridiales; | transport system | |
| | | f_Lachnospiraceae; | substrate-binding | |
| | | unclassified; unclassified | protein | |
| 71 | MH0131_GL00082 | d_Bacteria; p_Actinobacteria; | xylose isomerase | ↑ 1000.00 |
| | 68 | c_Actinobacteria; | | |
| | | o_Bifidobacteriales; | | |
| | | f_Bifidobacteriaceae; | | |
| | | g_Bifidobacterium; | | |
| | | s_Bifidobacterium adolescentis | | |
| 72 | V1.UC23- | d_Bacteria; p_Firmicutes; | glycerol kinase | ↑ 32.08701692 |
| | 1_GL0090652 | c_Clostridia; o_Clostridiales; | | |
| | | f_Eubacteriaceae; | | |
| | | g_Eubacterium; unclassified | | |
| 73 | MH0107_GL00940 | d_Bacteria; p_Firmicutes; | PTS system, N- | ↑ 22.77487489 |
| | 32 | c_Clostridia; o_Clostridiales; | acetylglucosamine- | |
| | | f_Ruminococcaceae; | specific IIB | |
| | | g_Faecalibacterium; | component | |
| | | s_Faecalibacterium prausnitzii | | |
| 74 | P02741 | Human | C-reactive protein | 0 |
| 75 | MH0420_GL00952 | d_Bacteria; p_Firmicutes; | lactose/L-arabinose | ↑ 12.62057153 |
| | 67 | c_Clostridia; o_Clostridiales; | transport system | |
| | | unclassified; unclassified; | substrate-binding | |
| | | unclassified | protein | |
| 76 | P27105 | Human | Erythrocyte band 7 | 0 |
| | | | integral membrane | |
| | | | protein | |
| 77 | 411483.FAEPRAA | d_Bacteria; p_Firmicutes; | pyruvate- | ↑ 1000.00 |
| | 2165_00243 | c_Clostridia; o_Clostridiales; | ferredoxin/flavodoxin | |
| | | f_Ruminococcaceae; | oxidoreductase | |
| | | g_Faecalibacterium; | | |
| | | s_Faecalibacterium prausnitzii | | |
| 78 | MH0149_GL00238 | d_Bacteria; p_Firmicutes; | raffinose/stachyose/ | ↑ 1000.00 |
| | 18 | c_Clostridia; o_Clostridiales; | melibiose transport | |
| | | f_Ruminococcaceae; | system substrate- | |
| | | g_Faecalibacterium; | binding protein | |
| | | s_Faecalibacterium prausnitzii | | |
| 79 | Q9Y6R7 | Human | IgGFc-binding | ↓ 0.303512739 |
| | | | protein | |
| 80 | NOF005_GL00693 | d_Bacteria; p_Firmicutes; | beta- | ↑ 12.78719114 |
| | 05 | c_Clostridia; o_Clostridiales; | fructofuranosidase | |
| | | unclassified; unclassified; | | |
| | | unclassified | | |
| 81 | O2.UC55- | d_Bacteria; p_Firmicutes; | multiple sugar | ↑ 20.32509166 |
| | 2_GL0069829 | c_Clostridia; o_Clostridiales; | transport system | |
| | | unclassified; unclassified; | substrate-binding | |
| | | unclassified | protein | |
| 82 | A0A0B4J1V0 | Human | Immunoglobulin | 0 |
| | | | heavy variable 3-15 | |
| 83 | P61626 | Human | Lysozyme C | ↓ 0.317780147 |
| 84 | P14555 | Human | Phospholipase A2, | ↓ 0.072089777 |
| | | | membrane associated | |
| 85 | MH0364_GL00600 | d_Bacteria; p_Firmicutes; | small subunit | ↑ 45.34709894 |
| | 24 | c_Clostridia; o_Clostridiales; | ribosomal protein S5 | |
| | | unclassified; unclassified; | | |
| | | unclassified | | |
| 86 | A0A0B4J231 | Human | Immunoglobulin | ↓ 0.259789454 |
| | | | lambda-like | |
| | | | polypeptide 5 | |
| 87 | Q9HD89 | Human | Resistin | 0 |
| 88 | P02748 | Human | Complement | 0 |
| | | | component C9 | |
| 89 | 763901136- | d_Bacteria; p_Firmicutes; | multiple sugar | ↑ 14.54517969 |
| | stool1_revised_{—} | c_Clostridia; o_Clostridiales; | transport system | |
| | C1085693_1_{—} | f_Lachnospiraceae; | ATP-binding protein | |
| | gene166508 | unclassified; unclassified | | |
| 90 | O2.UC2- | d_Bacteria; p_Actinobacteria; | enolase | ↑ 55.24026628 |
| | 1_GL0177495 | c_Actinobacteria; | | |
| | | o_Bifidobacteriales; | | |
| | | f_Bifidobacteriaceae; | | |
| | | g_Bifidobacterium; unclassified | | |
| 91 | MH0088_GL00462 | d_Bacteria; p_Firmicutes; | unknown | ↑ 1000.00 |
| | 32 | c_Clostridia; o_Clostridiales; | | |
| | | f_Eubacteriaceae; | | |
| | | g_Eubacterium; unclassified | | |
| 92 | Q02817 | Human | Mucin-2 | ↓ 0.248339613 |
| 93 | 160158126- | d_Bacteria; p_Firmicutes; | glutamate | ↑ 1000.00 |
| | stool2_revised_{—} | c_Clostridia; o_Clostridiales; | dehydrogenase | |
| | C609437_1_{—} | f_Lachnospiraceae; g_unknown; | (NADP+) | |
| | gene90477 | s_[Eubacterium] rectale | | |
| 94 | MH0087_GL00408 | d_Bacteria; p_Firmicutes; | molecular chaperone | ↑ 6.232038447 |
| | 66 | c_Clostridia; o_Clostridiales; | DnaK | |
| | | unclassified; unclassified; | | |
| | | unclassified | | |
| 95 | MH0087_GL00047 | d_Bacteria; p_Firmicutes; | small subunit | ↑ 9.611630723 |
| | 99 | c_Clostridia; o_Clostridiales; | ribosomal protein S4 | |
| | | unclassified; unclassified; | | |
| | | unclassified | | |
| 96 | P02763 | Human | Alpha-1-acid | 0 |
| | | | glycoprotein 1 | |
| 97 | MH0006_GL01997 | d_Bacteria; p_Firmicutes; | glycolate oxidase | ↑ 1000.00 |
| | 41 | c_Clostridia; o_Clostridiales; | | |
| | | unclassified; unclassified; | | |
| | | unclassified | | |
| 98 | 763840445- | d_Bacteria; p_Firmicutes; | large subunit | ↑ 5.03923981 |
| | stool2_revised_{—} | c_Clostridia; o_Clostridiales; | ribosomal protein | |
| | C906677_1_{—} | f_Lachnospiraceae; | L22 | |
| | gene54063 | unclassified; unclassified | | |
| 99 | MH0238_GL00278 | d_Bacteria; p_Firmicutes; | raffinose/stachyose/ | ↑ 1000.00 |
| | 53 | c_Clostridia; o_Clostridiales; | melibiose transport | |
| | | unclassified; unclassified; | system substrate- | |
| | | unclassified | binding protein | |
| 100 | MH0048_GL00451 | d_Bacteria; p_Firmicutes; | glutamate | ↑ 23.88539793 |
| | 61 | c_Clostridia; o_Clostridiales; | dehydrogenase | |
| | | f_Ruminococcaceae; | (NADP+) | |
| | | g_Ruminococcus; unclassified | | |
| 101 | A0M8Q6 | Human | Immunoglobulin | ↓ 0.150559 |
| | | | lambda constant 7 | |
| 102 | MH0329_GL01459 | d_Bacteria; p_Firmicutes; | glucose-6-phosphate | ↑ 20.75768401 |
| | 33 | c_Clostridia; o_Clostridiales; | isomerase | |
| | | f_Lachnospiraceae; | | |
| | | unclassified; unclassified | | |
| 103 | A0A0A0MS07 | Human | Immunoglobulin | 0 |
| | | | heavy constant | |
| | | | gamma 1 (Fragment) | |
| 104 | 158337416- | d_Bacteria; p_Firmicutes; | small subunit | ↑ 9.0403737 |
| | stool1_revised_{—} | c_Clostridia; o_Clostridiales; | ribosomal protein S2 | |
| | C1278792_1_{—} | f_Clostridiaceae; g_Clostridium; | | |
| | gene204797 | unclassified | | |
| 105 | V1.FI06_GL01719 | d_Bacteria; p_Firmicutes; | small subunit | ↑ 1000.00 |
| | 16 | c_Clostridia; o_Clostridiales; | ribosomal protein S4 | |
| | | f_Lachnospiraceae; | | |
| | | g_Anaerostipes; unclassified | | |
| 106 | P56470 | Human | Galectin-4 | ↓ 0.36537433 |
| 107 | 518635.BIFANG_{—} | d_Bacteria; p_Actinobacteria; | enolase | ↑ 1000.00 |
| | 03202 | c_Actinobacteria; | | |
| | | o_Bifidobacteriales; | | |
| | | f_Bifidobacteriaceae; | | |
| | | g_Bifidobacterium; unclassified | | |
| 108 | A0A0G2JMB2 | Human | Immunoglobulin | ↓ 0.526169248 |
| | | | heavy constant alpha | |
| | | | 2 (Fragment) | |
| 109 | O2.UC48- | d_Bacteria; p_Firmicutes; | acyl-CoA | ↑ 7.583216211 |
| | 0_GL0102815 | c_unknown; o_unknown; | dehydrogenase | |
| | | f_unknown; g_unknown; | | |
| | | s_Firmicutes bacterium CAG: 114 | | |
| 110 | HT14A_GL0029859 | d_Bacteria; p_Firmicutes; | butyryl-CoA | ↑ 1000.00 |
| | | c_unknown; o_unknown; | dehydrogenase | |
| | | f_unknown; g_unknown; | | |
| | | s_Firmicutes bacterium CAG: 114 | | |
| 111 | P02675 | Human | Fibrinogen beta chain | 0 |
| 112 | C9JEU5 | Human | Fibrinogen gamma | 0 |
| | | | chain | |
| 113 | MH0064_GL00253 | d_Bacteria; p_Bacteroidetes; | OmpA-OmpF porin, | ↑ 39.70136989 |
| | 57 | c_Bacteroidia; o_Bacteroidales; | OOP family | |
| | | f_Prevotellaceae; g_Prevotella; | | |
| | | s_Prevotella copri | | |
| 114 | A0A087WWT3 | Human | Serum albumin | ↓ 0.039991618 |
| 115 | MH0089_GL00675 | d_Bacteria; p_Firmicutes; | ketol-acid | ↑ 21.344027 |
| | 01 | c_Clostridia; o_Clostridiales; | reductoisomerase | |
| | | unclassified; unclassified; | | |
| | | unclassified | | |
| 116 | MH0087_GL00481 | d_Bacteria; p_Firmicutes; | L-ribulokinase | ↑ 20.39908966 |
| | 24 | c_Clostridia; o_Clostridiales; | | |
| | | f_Ruminococcaceae; | | |
| | | g_Ruminococcus; unclassified | | |
| 117 | MH0055_GL00318 | d_Bacteria; p_Firmicutes; | elongation factor Tu | ↑ 8.858505866 |
| | 24 | c_Clostridia; o_Clostridiales; | | |
| | | f_Ruminococcaceae; | | |
| | | g_Faecalibacterium; | | |
| | | s_Faecalibacterium prausnitzii | | |
| 118 | 158944319- | d_Bacteria; p_Firmicutes; | unknown | ↑ 28.88541996 |
| | stool1_revised_{—} | c_Clostridia; o_Clostridiales; | | |
| | C1053583_1_{—} | f_Clostridiaceae; g_Clostridium; | | |
| | gene115752 | s_Clostridium sp. CAG: 448 | | |
| 119 | A0A075B6P5 | Human | Immunoglobulin | 0 |
| | | | kappa variable 2-28 | |
| 120 | SZEY- | d_Bacteria; p_Firmicutes; | phosphoserine | ↑ 1000.00 |
| | 06A_GL0087252 | c_Clostridia; o_Clostridiales; | aminotransferase | |
| | | f_Lachnospiraceae; | | |
| | | g_Anaerostipes; unclassified | | |
| 121 | 764062976- | d_Bacteria; p_Firmicutes; | pyruvate, | \| 1000.00 |
| | stool2_revised_{—} | c_Clostridia; o_Clostridiales; | orthophosphate | |
| | scaffold46358_1_{—} | f_Lachnospiraceae; unclassified; | dikinase | |
| | gene78650 | unclassified | | |
| 122 | MH0062_GL00653 | d_Bacteria; p_Firmicutes; | PTS system, N- | ↑ 1000.00 |
| | 18 | c_Clostridia; o_Clostridiales; | acetylglucosamine- | |
| | | unclassified; unclassified; | specific IIB | |
| | | unclassified | component | |
| 123 | Q14002 | Human | Carcinoembryonic | ↓ 0.093216867 |
| | | | antigen-related cell | |
| | | | adhesion molecule 7 | |
| 124 | A0A286YEY4 | Human | Immunoglobulin | 0 |
| | | | heavy constant | |
| | | | gamma 2 (Fragment) | |
| 125 | P08861 | Human | Chymotrypsin-like | ↓ 0.075567809 |
| | | | elastase family | |
| | | | member 3B | |
| 126 | P0DOY2 | Human | Immunoglobulin | ↓ 0.028287899 |
| | | | lambda constant 2 | |
| 127 | MH0230_GL01539 | d_Bacteria; p_Firmicutes; | PTS system, | ↑ 1000.00 |
| | 29 | c_Clostridia; o_Clostridiales; | mannos-specific IIA | |
| | | unclassified; unclassified; | component | |
| | | unclassified | | |
| 128 | V1.UC51- | d_Bacteria; p_Firmicutes; | ribose transport | ↑ 1000.00 |
| | 4_GL0052281 | c_Clostridia; o_Clostridiales; | system substrate- | |
| | | f_Ruminococcaceae; | binding protein | |
| | | g_Ruminococcus; unclassified | | |
| 129 | MH0127_GL00481 | d_Bacteria; p_Firmicutes; | phosphoenolpyruvate | ↑ 1000.00 |
| | 00 | c_Clostridia; o_Clostridiales; | carboxykinase (ATP) | |
| | | unclassified; unclassified; | | |
| | | unclassified | | |
| 130 | P08217 | Human | Chymotrypsin-like | ↓ 0.410267674 |
| | | | elastase family | |
| | | | member 2A | |
| 131 | MH0358_GL01030 | d_Bacteria; p_Firmicutes; | glutamate | ↑ 6.820041208 |
| | 72 | c_Clostridia; o_Clostridiales; | dehydrogenase | |
| | | f_Ruminococcaceae; | (NADP+) | |
| | | g_Ruminococcus; unclassified | | |
| 132 | Q08380 | Human | Galectin-3-binding | ↓ 0.088411784 |
| | | | protein | |
| 133 | MH0417_GL01005 | d_Bacteria; p_Firmicutes; | acetyl-CoA | ↑ 1000.00 |
| | 60 | c_Clostridia; o_Clostridiales; | decarbonylase/ | |
| | | unclassified; unclassified; | synthase, CODH/ACS | |
| | | unclassified | comples subunit | |
| | | | gamma | |
| 134 | MH0227_GL01531 | d_Bacteria; p_Firmicutes; | 5′-nucleotidase | ↑ 1000.00 |
| | 99 | c_Clostridia; o_Clostridiales; | | |
| | | f_Ruminococcaceae; | | |
| | | g_Faecalibacterium; unclassified | | |
| 135 | 657314.CK5_22290 | d_Bacteria; p_Firmicutes; | raffinose/stachyose/ | \|1000.00 |
| | | c_Clostridia; o_Clostridiales; | melibiose transport | |
| | | unclassified; unclassified; | system substrate- | |
| | | unclassified | binding protein | |
| 136 | MH0363_GL01548 | d_Bacteria; p_Firmicutes; | raffinose/stachyose/ | ↑ 1000.00 |
| | 63 | c_Clostridia; o_Clostridiales; | melibiose transport | |
| | | unclassified; unclassified; | system substrate- | |
| | | unclassified | binding protein | |
| 137 | P19961 | Human | Alpha-amylase 2B | ↑ 2.386953261 |
| 138 | MH0111_GL01184 | d_Bacteria; p_Firmicutes; | simple sugar transport | ↑ 1000.00 |
| | 09 | c_Clostridia; o_Clostridiales; | system substrate- | |
| | | f_Lachnospiraceae; unclassified; | binding protein | |
| | | unclassified | | |
| 139 | MH0110_GL00031 | d_Bacteria; p_Firmicutes; | multiple sugar | ↑ 1000.00 |
| | 28 | c_Clostridia; o_Clostridiales; | transport system | |
| | | f_Ruminococcaceae; | ATP-binding protein | |
| | | g_Faecalibacterium; | | |
| | | s_Faecalibacterium prausnitzii | | |
| 140 | A0A024R6I7 | Human | Alpha-1-antitrypsin | ↓ 0.505976896 |
| 141 | 158944319- | d_Bacteria; p_Firmicutes; | ketol-acid | ↑ 1000.00 |
| | stool2_revised_{—} | c_Clostridia; o_Clostridiales; | reductoisomerase | |
| | C1470499_1_{—} | unclassified; unclassified; | | |
| | gene61965 | unclassified | | |
| 142 | MH0184_GL01184 | d_Bacteria; p_Firmicutes; | raffinose/stachyose/ | ↑ 10.56645944 |
| | 71 | c_Clostridia; o_Clostridiales; | melibiose transport | |
| | | f_Ruminococcaceae; | system substrate- | |
| | | g_Subdoligranulum; | binding protein | |
| | | s_Subdoligranulum sp. | | |
| | | APC924/74 | | |
| 143 | MH0188_GL00881 | d_Bacteria; p_Firmicutes; | pyruvate, | ↑ 6.959656766 |
| | 09 | c_Clostridia; o_Clostridiales; | orthophosphate | |
| | | f_Eubacteriaceae; g_Eubacterium; | dikinase | |
| | | unclassified | | |
| 144 | V1.FI35_GL01073 | d_Bacteria; p_Firmicutes; | pyruvate, | ↑ 1000.00 |
| | 38 | c_Clostridia; o_Clostridiales; | orthophosphate | |
| | | unclassified; unclassified; | dikinase | |
| | | unclassified | | |
| 145 | O2.UC48- | d_Bacteria; p_Firmicutes; | acetylornithine/N- | ↑ 1000.00 |
| | 0_GL0103416 | c_Clostridia; o_Clostridiales; | succinyldiamino- | |
| | | f_Ruminococcaceae; g_unknown; | pimelate aminotrans- | |
| | | s_Ruminococcaceae bacterium | ferase | |
| | | KLE1738 | | |
| 146 | P01009 | Human | Alpha-1-antitrypsin | ↓ 0 |
| 147 | 159268001- | d_Bacteria; p_Firmicutes; | acetyl-CoA | ↑ 9.473922743 |
| | stool2_revised_{—} | c_Clostridia; o_Clostridiales; | decarbonylase/synthase, | |
| | scaffold33645_1_{—} | f_Ruminococcaceae; | CODH/ACS complex | |
| | gene143773 | g_Ruminococcus; unclassified | subunit delta | |
| 148 | MH0371_GL00876 | d_Bacteria; p_Firmicutes; | phosphoglycerate | ↑ 18.70031035 |
| | 22 | c_Clostridia; o_Clostridiales; | kinase | |
| | | unclassified; unclassified; | | |
| | | unclassified | | |
| 149 | MH0188_GL00283 | d_Bacteria; p_Firmicutes; | lactose/L-arabinose | ↑ 9.113003783 |
| | 15 | c_Clostridia; o_Clostridiales; | transport system | |
| | | unclassified; unclassified; | substrate-binding | |
| | | unclassified | protein | |
| 150 | O75594 | Human | Peptidoglycan | 0 |
| | | | recognition protein 1 | |
| 151 | MH0199_GL01909 | d_Bacteria; p_Firmicutes; | PTS system, | ↑ 1000.00 |
| | 08 | c_Clostridia; o_Clostridiales; | mannose-specific IID | |
| | | unclassified; unclassified; | component | |
| | | unclassified | | |
| 152 | ED14A_GL0062492 | d_Bacteria; p_Firmicutes; | C4-dicarboxylate- | ↑1000.00 |
| | | c_Clostridia; o_Clostridiales; | binding protein DctP | |
| | | f_Ruminococcaceae; | | |
| | | g_Faecalibacterium; | | |
| | | s_Faecalibacterium prausnitzii | | |
| 153 | E9PGN7 | Human | Plasma protease C1 | 0 |
| | | | inhibitor | |
| 154 | MH0088_GL01281 | d_Bacteria; p_Firmicutes; | lactose/L-arabinose | ↑ 18.88639806 |
| | 77 | c_Clostridia; o_Clostridiales; | transport system | |
| | | f_Lachnospiraceae; g_Blautia; | substrate-binding | |
| | | unclassified | protein | |
| 155 | MH0108_GL00570 | d_Bacteria; p_Firmicutes; | 3-hydroxybutyryl- | ↑ 1000.00 |
| | 76 | c_Clostridia; o_Clostridiales; | CoA dehydrogenase | |
| | | f_Ruminococcaceae; | | |
| | | g_Faecalibacterium; | | |
| | | s_Faecalibacterium prausnitzii | | |
| 156 | 160603188- | d_Bacteria; p_Firmicutes; | ethanolamine | ↑ 5.153152742 |
| | stool1_revised_{—} | c_Clostridia; o_Clostridiales; | utilization protein | |
| | C908439_1_{—} | unclassified; unclassified; | EutM | |
| | gene130220 | unclassified | | |
| 157 | Q6UWV6 | Human | Ectonucleotide | ↑ 3.563838475 |
| | | | pyrophosphatase/ | |
| | | | phosphodiesterase | |
| | | | family member 7 | |
| 158 | Q8WWU7 | Human | Intelectin-2 | ↓ 0.518184363 |
| 159 | MH0086_GL00772 | d_Bacteria; p_Firmicutes; | butyryl-CoA | ↑ 1000.00 |
| | 08 | c_Clostridia; o_Clostridiales; | dehydrogenase | |
| | | f_Clostridiaceae; unclassified; | | |
| | | unclassified | | |
| 160 | P55259 | Human | Pancreatic secretory | ↓ 0.398403752 |
| | | | granule membrane | |
| | | | major glycoprotein | |
| | | | GP2 | |
| 161 | 159268001- | d_Bacteria; p_Firmicutes; | acetyl-CoA C- | ↑ 19.24322786 |
| | stool2_revised_{—} | c_Clostridia; o_Clostridiales; | acetyltransferase | |
| | scaffold1608_1_{—} | f_Ruminococcaceae; | | |
| | gene43841 | g_Faecalibacterium; | | |
| | | s_Faecalibacterium prausnitzii | | |
| 162 | MH0086_GL00986 | d_Bacteria; p_Firmicutes; | peptide/nickel | ↑ 21.88877191 |
| | 87 | c_Clostridia; o_Clostridiales; | transport system | |
| | | unclassified; unclassified; | substrate-binding | |
| | | unclassified | protein | |
| 163 | MH0301_GL00221 | d_Bacteria; p_Firmicutes; | elongation factor Tu | ↑ 16.94864584 |
| | 39 | c_Clostridia; o_Clostridiales; | | |
| | | unclassified; unclassified; | | |
| | | unclassified | | |
| 164 | 158337416- | d_Bacteria; p_Firmicutes; | ketol-acid | ↑ 20.13816158 |
| | stool1_revised_{—} | c_Clostridia; o_Clostridiales; | reductoisomerase | |
| | C1186490_1_{—} | f_Lachnospiraceae; unclassified; | | |
| | gene63866 | unclassified | | |
| 165 | MH0088_GL00537 | d_Bacteria; p_Firmicutes; | oxaloacetate | ↑ 12.82137202 |
| | 47 | c_Clostridia; o_Clostridiales; | decarboxylase (Na+ | |
| | | unclassified; unclassified; | extruding) subunit | |
| | | unclassified | alpha | |
| 166 | 160218816- | d_Bacteria; p_Firmicutes; | L-fucose/D-arabinose | ↑ 11.01160309 |
| | stool1_revised_{—} | c_Clostridia; o_Clostridiales; | isomerase | |
| | scaffold24109_1_{—} | unclassified; unclassified; | | |
| | gene74537 | unclassified | | |
| 167 | V1.UC33- | d_Bacteria; p_Firmicutes; | fructoselysine 6- | \| 1000.00 |
| | 0_GL0031426 | c_Clostridia; o_Clostridiales; | phosphate deglycase | |
| | | f_Ruminococcaceae; | | |
| | | g_Faecalibacterium; | | |
| | | s_Faecalibacterium prausnitzii | | |
| 168 | O2.UC36- | d_Bacteria; p_Firmicutes; | butyryl-CoA | ↑ 1000.00 |
| | 0_GL0022629 | c_Clostridia; o_Clostridiales; | dehydrogenase | |
| | | f_Eubacteriaceae; g_Eubacterium; | | |
| | | unclassified | | |
| 169 | A0A2R8Y793 | Human | Actin, cytoplasmic 1 | 0 |
| | | | (Fragment) | |
| 170 | DLF012_GL00395 | d_Bacteria; p_Firmicutes; | multiple sugar | ↑ 14.18558156 |
| | 73 | c_Clostridia; o_Clostridiales; | transport system | |
| | | unclassified; unclassified; | substrate-binding | |
| | | unclassified | protein | |
| 171 | MH0188_GL00126 | d_Bacteria; p_Firmicutes; | raffinose/stachyose/ | ↑ 1000.00 |
| | 88 | c_Clostridia; o_Clostridiales; | melibiose transport | |
| | | f_Eubacteriaceae; g_Eubacterium; | system substrate- | |
| | | s_[Eubacterium] hallii | binding protein | |
| 172 | MH0233_GL01085 | d_Bacteria; p_Firmicutes; | lactose/L-arabinose | ↑ 1000.00 |
| | 03 | c_Clostridia; o_Clostridiales; | transport system | |
| | | unclassified; unclassified; | substrate-binding | |
| | | unclassified | protein | |
| 173 | V1.UC48- | d_Bacteria; p_Firmicutes; | aspartyl-tRNA | ↑ 1000.00 |
| | 0_GL0002861 | c_Clostridia; o_Clostridiales; | synthetase | |
| | | f_Lachnospiraceae; unclassified; | | |
| | | unclassified | | |
| 174 | MH0422_GL00905 | d_Bacteria; p_Firmicutes; | molecular chaperone | ↑ 1000.00 |
| | 15 | c_Clostridia; o_Clostridiales; | DnaK | |
| | | f_Lachnospiraceae; | | |
| | | g_Anaerostipes; unclassified | | |
| 175 | MH0087_GL00127 | d_Bacteria; p_Firmicutes; | Na+-translocating | ↑ 1000.00 |
| | 28 | c_Clostridia; o_Clostridiales; | ferredoxin:NAD+ | |
| | | f_Ruminococcaceae; | oxidoreductase | |
| | | g_Ruminococcus; unclassified | subunit C | |
| 176 | MH0089_GL00260 | d_Bacteria; p_Firmicutes; | elongation factor G | ↑ 1000.00 |
| | 62 | c_Clostridia; o_Clostridiales; | | |
| | | f_Lachnospiraceae; unclassified; | | |
| | | unclassified | | |
| 177 | P21796 | Human | Voltage-dependent | ↓ 0.038616435 |
| | | | anion-selective | |
| | | | channel protein 1 | |
| 178 | MH0012_GL01041 | d_Bacteria; p_Firmicutes; | phosphoenolpyruvate | ↑ 1000.00 |
| | 43 | c_Clostridia; o_Clostridiales; | carboxykinase (ATP) | |
| | | f_Lachnospiraceae; unclassified; | | |
| | | unclassified | | |
| 179 | Q10588 | Human | ADP-ribosyl | 0 |
| | | | cyclase/cyclic ADP- | |
| | | | ribose hydrolase 2 | |
| 180 | P31997 | Human | Carcinoembryonic | 0 |
| | | | antigen-related cell | |
| | | | adhesion molecule 8 | |
| 181 | P06312 | Human | Immunoglobulin | 0 |
| | | | kappa variable 4-1 | |
| 182 | 158742018- | d_Bacteria; p_Bacteroidetes; | fructose-bisphosphate | 0 |
| | stool1_revised_{—} | c_Bacteroidia; o_Bacteroidales; | aldolase, class II | |
| | scaffold17184_1_{—} | f_Tannerellaceae; | | |
| | gene50964 | g_Parabacteroides; unclassified | | |
| 183 | MH0131_GL01038 | d_Bacteria; p_Firmicutes; | ketol-acid | \| 1000.00 |
| | 03 | c_Clostridia; o_Clostridiales; | reductoisomerase | |
| | | unclassified; unclassified; | | |
| | | unclassified | | |
| 184 | 515619.EUBREC_{—} | d_Bacteria; p_Firmicutes; | pyruvate- | ↑ 1000.00 |
| | 1472 | c_Clostridia; o_Clostridiales; | ferredoxin/flavodoxin | |
| | | f_Lachnospiraceae; unclassified; | oxidoreductase | |
| | | unclassified | | |
| 185 | MH0156_GL00554 | d_Bacteria; p_Firmicutes; | carbon starvation | ↑ 1000.00 |
| | 57 | c_Clostridia; o_Clostridiales; | protein | |
| | | f_Ruminococcaceae; | | |
| | | g_Faecalibacterium; | | |
| | | s_Faecalibacterium prausnitzii | | |
| 186 | MH0005_GL00161 | d_Bacteria; p_Bacteroidetes; | Ca-activated chloride | ↑ 1000.00 |
| | 99 | c_Bacteroidia; o_Bacteroidales; | channel homolog | |
| | | f_Prevotellaceae; g_Prevotella; | | |
| | | s_Prevotella copri | | |
| 187 | DOM013_GL0034 | d_Bacteria; p_Firmicutes; | acetyl-CoA C- | ↑ 1000.00 |
| | 020 | c_Clostridia; o_Clostridiales; | acetyltransferase | |
| | | f_Ruminococcaceae; | | |
| | | g_Faecalibacterium; | | |
| | | s_Faecalibacterium prausnitzii | | |
| 188 | P11215 | Human | Integrin alpha-M | 0 |
| 189 | A0A2R8Y7C0 | Human | Hemoglobin subunit | 0 |
| | | | alpha (Fragment) | |
| 190 | MH0359_GL01138 | d_Bacteria; p_Firmicutes; | elongation factor Tu | ↑ 1000.00 |
| | 01 | c_Clostridia; o_Clostridiales; | | |
| | | f_Ruminococcaceae; | | |
| | | g_Faecalibacterium; | | |
| | | s_Faecalibacterium prausnitzii | | |
| 191 | O2.UC32- | d_Bacteria; p_Firmicutes; | L-fucose mutarotase | ↑ 1000.00 |
| | 0_GL0072495 | c_Clostridia; o_Clostridiales; | | |
| | | unclassified; unclassified; | | |
| | | unclassified | | |
| 192 | P80511 | Human | Protein S100-A12 | 0 |
| 193 | V1.FI05_GL01145 | d_Bacteria; p_Actinobacteria; | raffinose/stachyose/ | ↑ 1000.00 |
| | 31 | c_Actinobacteria; | melibiose transport | |
| | | o_Bifidobacteriales; | system substrate- | |
| | | f_Bifidobacteriaceae; | binding protein | |
| | | g_Bifidobacterium; | | |
| | | s_Bifidobacterium | | |
| | | pseudocatenulatum | | |
| 194 | 764143897- | d_Bacteria; p_Firmicutes; | oxaloacetate | ↑ 1000.00 |
| | stool1_revised_{—} | c_Clostridia; o_Clostridiales; | decarboxylase (Na+ | |
| | scaffold20558_1_{—} | f_Lachnospiraceae; unclassified; | extruding) subunit | |
| | gene103709 | unclassified | alpha | |
| 195 | 763860675- | d_Bacteria; p_Firmicutes; | pyruvate- | 17.65909965 |
| | stool1_revised_{—} | c_Clostridia; o_Clostridiales; | ferredoxin/flavodoxin | |
| | scaffold52092_1_{—} | f_Ruminococcaceae; | oxidoreductase | |
| | gene192072 | g_Ruminococcus; unclassified | | |
| 196 | O2.UC35- | d_Bacteria; p_Firmicutes; | multiple sugar | ↑ 1000.00 |
| | 0_GL0038446 | c_Clostridia; o_Clostridiales; | transport system | |
| | | f_Eubacteriaceae; g_Eubacterium; | substrate-binding | |
| | | s_[Eubacterium] eligens | protein | |
| 197 | P28676 | Human | Grancalcin | 0 |
| 198 | O2.UC4- | d_Bacteria; p_Firmicutes; | | ↑ 1000.00 |
| | 1_GL0180535 | c_Erysipelotrichia; | | |
| | | o_Erysipelotrichales; | | |
| | | f_Erysipelotrichaceae; | | |
| | | unclassified; unclassified | | |
| 199 | 290338.CKO_04745 | d_Bacteria; p_Proteobacteria; | elongation factor Tu | 0.047695655 |
| | | c_Gammaproteobacteria; | | |
| | | o_Enterobacterales; | | |
| | | f_Enterobacteriaceae; | | |
| | | unclassified; unclassified | | |
| 200 | Q06141 | Human | Regenerating islet- | 0.012746588 |
| | | | derived protein 3-alpha | |

TABLE C
Examples of Metaproteome Features from a Human Host

| | | | Control vs CDI |
|---|---|---|---|
| Rank # by | | | or other |
| ANOVA F- | | | pathogen |
| Values | Feature ID | Feature_function | Fold change |
| 1 | P01833 | Polymeric immunoglobulin receptor | ↓ 0.212667632 |
| 2 | E7EQB2 | Lactotransferrin (Fragment) | ↓ 0.060725156 |
| 3 | P05164 | Myeloperoxidase | ↓ 0.049038368 |
| 4 | P05109 | Protein S100-A8 | ↓ 0.074193038 |
| 12 | Q9BYE9 | Cadherin-related family member 2 | ↑ 8.002048656 |
| 7 | P01619 | Immunoglobulin kappa variable 3-20 | ↓ 0.059065469 |
| 8 | P11678 | Eosinophil peroxidase | ↓ 0.120582681 |
| 9 | P08246 | Neutrophil elastase | ↓ 0.038622247 |
| 14 | A0A1B0GUU9 | Immunoglobulin heavy constant mu | ↓ 0.053254188 |
| | | (Fragment) | |
| 15 | P24158 | Myeloblastin | ↓ 0.087757606 |
| 16 | P08311 | Cathepsin G | ↓ 0.034043287 |
| 17 | Q9UGM3 | Deleted in malignant brain tumors 1 | ↓ 0.288744122 |
| | | protein | |
| 19 | P06702 | Protein S100-A9 | ↓ 0.067195481 |
| 24 | P20160 | Azurocidin | ↓ 0.019640472 |
| 23 | Q8WWA0 | Intelectin-1 | ↓ 0.324559777 |
| 27 | A8K714 | Calcium-activated chloride channel | ↓ 0.449866903 |
| | | regulator 1 | |
| 32 | Q9H3R2 | Mucin-13 | ↓ 0.077154852 |
| 28 | A0A286YEY1 | Immunoglobulin heavy constant alpha | ↓ 0.527370307 |
| | | 1 (Fragment) | |
| 31 | P01834 | Immunoglobulin kappa constant | ↓ 0.39484137 |
| 38 | P02814 | Submaxillary gland androgen-regulated | ↑ 9.698731185 |
| | | protein 3B | |
| 33 | D6RD17 | Immunoglobulin J chain (Fragment) | ↓ 0.246551531 |
| 34 | P01024 | Complement C3 | ↓ 0 |
| 39 | P01023 | Alpha-2-macroglobulin | ↓ 0.094877837 |
| 43 | A0A0C4DGB6 | Serum albumin | ↓ 0 |
| 59 | P15144 | Aminopeptidase N | ↓ 0.205775187 |
| 56 | P13688 | Carcinoembryonic antigen-related cell | ↓ 0.086058183 |
| | | adhesion molecule 1 | |
| 57 | P12724 | Eosinophil cationic protein | ↓ 0.025432181 |
| 55 | P59665 | Neutrophil defensin 1 | ↓ 0.169473785 |
| 60 | P13727 | Bone marrow proteoglycan | ↓ 0 |
| 64 | P80188 | Neutrophil gelatinase-associated | ↓ 0.03800983 |
| | | lipocalin | |
| 62 | A0A024R0K5 | Carcinoembryonic antigen-related cell | ↓ 0.29197593 |
| | | adhesion molecule 5, isoform CRA_a | |
| 65 | P05451 | Lithostathine-l-alpha | ↓ 0.096470566 |
| 74 | P02741 | C-reactive protein | ↓ 0 |
| 76 | P27105 | Erythrocyte band 7 integral membrane | ↓ 0 |
| | | protein | |
| 86 | A0A0B4J231 | Immunoglobulin lambda-like | ↓ 0.229498622 |
| | | polypeptide 5 | |
| 84 | P14555 | Phospholipase A2, membrane | ↓ 0.040951112 |
| | | associated | |
| 79 | Q9Y6R7 | IgGFc-binding protein | ↓ 0.327053228 |
| 82 | A0A0B4J1V0 | Immunoglobulin heavy variable 3-15 | ↓ 0 |
| 87 | Q9HD89 | Resistin | ↓ 0 |
| 88 | P02748 | Complement component C9 | ↓ 0 |
| 130 | P08217 | Chymotrypsin-like elastase family | ↓ 0.343571258 |
| | | member 2A | |
| 101 | A0M8Q6 | Immunoglobulin lambda constant 7 | ↓ 0.133952186 |
| 83 | P61626 | Lysozyme C | ↓ 0.345140701 |
| 96 | P02763 | Alpha-1-acid glycoprotein 1 | ↓ 0 |
| 92 | Q02817 | Mucin-2 | ↓ 0.263044648 |
| 103 | A0A0A0MS07 | Immunoglobulin heavy constant | ↓ 0 |
| | | gamma 1 (Fragment) | |
| 108 | A0A0G2JMB2 | Immunoglobulin heavy constant alpha | ↓ 0.52721189 |
| | | 2 (Fragment) | |
| 111 | P02675 | Fibrinogen beta chain | ↓ 0 |
| 123 | Q14002 | Carcinoembryonic antigen-related cell | ↓ 0.068828388 |
| | | adhesion molecule 7 | |
| 112 | C9JEU5 | Fibrinogen gamma chain | ↓ 0 |

In specific embodiments, Table C encompasses human host metaproteome features that allows prediction of clinical outcome for the host individual whether or not the individual has had diarrhea (including diarrhea suspected of being related to antibiotics and/or CDI or another pathogenic microbe) and/or has had antibiotics. Embodiments of the disclosure provide for identification of individuals that will be responsive to a particular treatment, including at least FMT.

### V. Diarrheal Diseases and Samples

Particular embodiments concern the methods and systems of detecting and/or measuring features indicative of a diarrheal disease in an individual. The diarrheal disease may be any disease with symptomatic diarrhea, including antibiotic-associated diarrhea (AAD), a Clostridioides infection, a functional gastrointestinal disorder, for example. AAD may be caused by an antibiotic such as cephalosporin or a relevant analog, penicillin or a relevant analog. AAD may be caused by an imbalance of commensal and pathogenic bacteria in the gastrointestinal tract of the individual.

Food allergies (cow's milk, soy, cereal grains, eggs, and seafood) and intolerances (lactose or fructose or sugar alcohols), digestive tract diseases, or infections may cause diarrhea in an individual. Three types of infections that cause diarrhea include viral infections (for example, norovirus and rotavirus); bacterial infections (such as Campylobacter, Escherichia coli (E. coli ), Salmonella, and Shigella); and parasitic infections (such as Cryptosporidium enteritis, Entamoeba histolytica, and Giardia lamblia). Several types of bacteria can enter the body through contaminated food or water and cause diarrhea. Parasites can enter the body through food or water and settle in the digestive tract.

In some cases wherein antibiotics and/or antimicrobials are the cause of diarrhea, broad-spectrum antibiotics may be the cause, such as cleocin (clindamycin), certain types of penicillin, and cephalosporins. Individuals that are hospitalized or in nursing homes may be subject to methods of the disclosure because they have diarrhea or are prone to CDI and other types of infection that causes diarrhea. Individuals that are on a cruise ship or will be on a cruise ship may be subjected to methods of the disclosure to distinguish their susceptibility to CDI versus norovirus and/or rotavirus infection.

Samples may or may not be obtained by the same individual that performs the method steps. Fecal samples may be provided by the individual seeking treatment or diagnosis, or fecal samples may be obtained by a medical practitioner.

### VI. Detection Assays

One of more features encompassed herein may be detected based on their form being nucleic acid, protein, or small molecule, such as a metabolite.

A. Nucleic Acid Detection

Embodiments of the disclosure include methods of detection of particular 16S rRNA sequences, including that of any one of the features of Table A, for example. In cases wherein the nucleic acid of more than one feature is analyzed, the separate nucleic acids may or may not be analyzed simultaneously.

For amplification and detection of sequences found in the appropriate 16S rRNA sequences (which include 16S rRNA and genes encoding 16S rRNA), oligonucleotides may be designed and utilized that act as amplification oligomers and detection probes and that are specific and unique for the particular feature. With respect to oligonucleotides that may be utilized for directed hybridization and subsequent analysis, specific sequences may be selected, the oligonucleotides synthesized in vitro, and then optionally characterized by determining the Tm and hybridization characteristics of the oligonucleotides with complementary target sequences using standard laboratory methods. Desired oligonucleotides are utilized in amplification reactions with 16S rRNA purified from a sample. Prior to clinical use, the relative efficiencies of different combinations of amplification oligonucleotides may be determined by detecting the amplified products of the amplification reactions, generally by binding a labeled probe to the amplified products and detecting the relative amount of signal that indicates the amount of amplified product made.

Specific oligonucleotides may be designed to amplify and detect target sequences in 16S rRNA or DNA encoding 16S rRNA from a sample. In some cases, multiple sets of amplification and detection oligonucleotides may be utilized.

Amplification oligonucleotides include those that may function as primers. Amplification oligonucleotides may be modified by synthesizing the oligomer with a 3′ blocked end. The blocked oligomers may be used in a single primer transcription associated amplification reaction, i.e., functioning as blocking molecules or promoter provider oligomers.

In particular embodiments, one or more of the 16S rRNA features are identified using polymerase chain reaction. In specific embodiments, a multiplex PCR assay is utilized. In specific cases, primer pairs directed to respective, multiple 16S rRNA features are utilized substantially simultaneously against nucleic acid from a sample from an individual. In specific embodiments, quantitative PCR is utilized. In specific embodiments, PCR of any kind, quantitative isothermal DNA amplification, in situ hybridization, and/or next generation sequencing is utilized

B. Protein and Metabolite Detection

In particular embodiments, the one or more features are in the form of protein, and assays are performed to measure the level of the respective protein(s). A particular protein feature may be analyzed solely for a method, or multiple proteins may be analyzed either separately or simultaneously. Protein features may originate from the host or from a microbe in the host.

Protein detection methods may utilize spectrometry methods (such as high performance liquid chromatography or mass spectrometry) or antibody-based methods, such as enzyme-linked immunosorbent assays (ELISA) or western blot. The term “antibody” is used to refer to any antibody-like molecule that has an antigen binding region, and includes antibody fragments such as Fab′, Fab, F(ab′)2, single domain antibodies (DAB s), Fv, scFv (single chain Fv), and the like.

In specific embodiments, metabolites are analyzed by mass spectrometry, ELISA, chromatography, or a combination thereof, and proteins are analyzed by mass spectrometry, ELISA, chromatography, Western blotting, immunoprecipitation, immunoelectrophoresis, or a combination thereof.

### VII. Algorithms

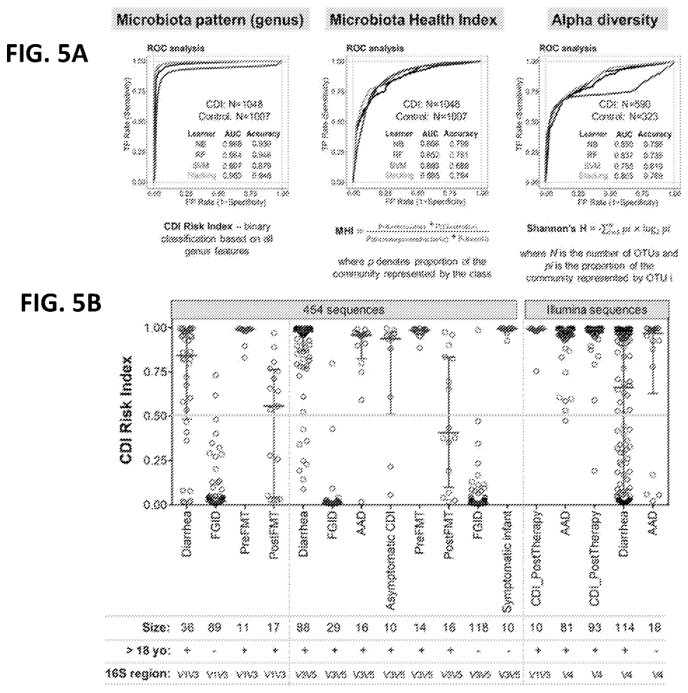

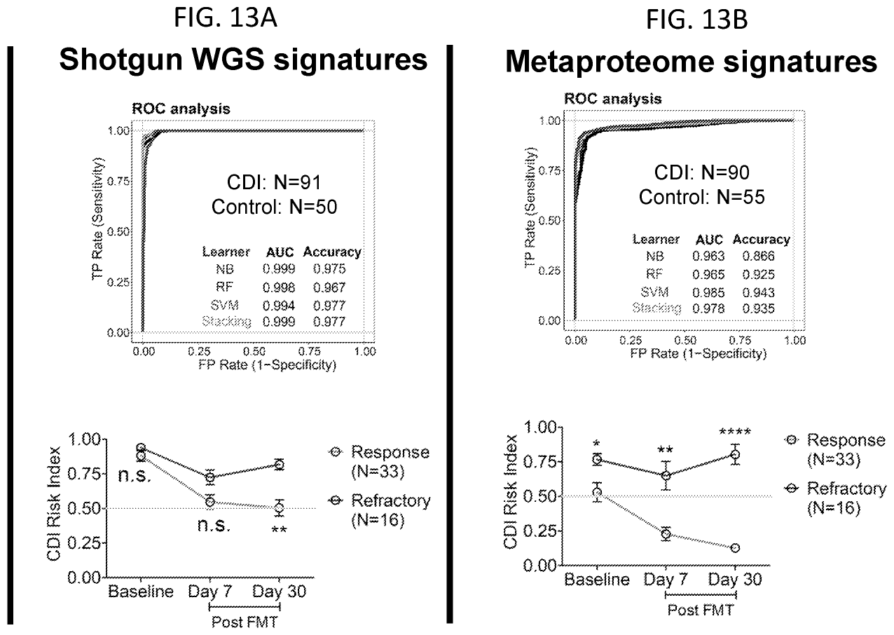

In particular embodiments, an algorithm is employed to compute information of one or more various features from a sample from an individual. In specific embodiments, the microbiome and/or metaproteome feature data of a training set were generated from 16S rRNA gene amplicon sequencing data and shotgun metaproteome data analyzed by bioinformatics pipelines (FIGS. 5 and 13).

The construction of supervised learning feature was achieved by using individual learning algorithms (Naïve Bayes, Random Forest, Support Vector Machine etc.) or a combination of learning algorithms for learning the feature patterns of the training set with the balanced size of CDI (or other pathogens) and Control samples. The default cut-off of such binary classification is set to 0.50 during the training process.

The feature data of a clinical sample (stool specimen) generated through bioinformatics pipelines is analyzed by the feature. The feature generates a class (either CDI (or other pathogens) or Control) and a prediction score ranging from 0 to 1 that is linked to the class. A score higher than 0.50 indicates the CDI (or other pathogen) state of the clinical sample, while a score lower than 0.50 indicates the Control state of the clinical sample.

### VIII. Kits

One can recognize that based on the methods described herein, detection reagents, kits, and/or systems can be utilized to detect the features related to the disease signature for diagnosing an individual (the detection either individually or in combination). The reagents can be combined into at least one of the established formats for kits and/or systems as known in the art. As used herein, the terms “kits” and “systems” refer to embodiments such as combinations of at least one nucleic acid detection reagent, at least one metabolite detection reagent, and/or at least one protein detection reagent. Non-limiting examples of nucleic acid reagents include at least one nucleic acid isolation reagent, at least one selective oligonucleotide probe, at least one sequencing reagent, and/or at least one PCR primer. Non-limiting examples of metabolite detection reagents include at least one metabolite extraction reagent, at least one enzyme capable of detecting specific metabolites, at least one chromatography reagent, and/or at least one mass spectrometry reagent. Non-limiting examples of protein detection reagents include at least one protein isolation reagent, at least one protein-specific antibody, at least one chromatography reagent, and/or at least one mass spectrometry reagent.

The kits could also contain other reagents, chemicals, buffers, enzymes, packages, containers, electronic hardware components, etc. The kits/systems could also contain packaged sets of PCR primers, oligonucleotides, arrays, beads, or other detection reagents. Any number of probes could be implemented for a detection array. In some embodiments, the detection reagents and/or the kits/systems are paired with chemiluminescent or fluorescent detection reagents. Particular embodiments of kits/systems include the use of electronic hardware components, such as DNA chips or arrays, or microfluidic systems, for example. In some embodiments, the kit provides a platform for performing mass spectrometry on the sample to measure the features disclosed herein. Mass spectrometry methods may include MALDI-TOF, LC-MS, GC-MS, IC-MS, for example. In particular embodiments, the kit provides a platform for performing an enzyme-linked immunosorbent assay (ELISA) to measure the levels of classifiers disclosed herein in a sample. In specific embodiments, the kit also comprises one or more therapeutic or prophylactic interventions in the event the individual is determined to be in need of.

### EXAMPLES

The following examples are included to demonstrate certain non-limiting aspects of the disclosure. It should be appreciated by those of skill in the art that the techniques disclosed in the examples that follow represent techniques discovered by the inventors to function well in the practice of the disclosed subject matter. However, those of skill in the art should, in light of the present disclosure, appreciate that many changes can be made in the specific embodiments that are disclosed and still obtain a like or similar result without departing from the spirit and scope of the disclosed subject matter.

### Example 1

### Predicting Patient Susceptibility to C. difficile Infection: Functional Insights into Microbiome Dysbiosis and Host Signatures

The present example includes data from CDI patients that provides one approach that can be extended to interrogate common host-microbiota susceptibility features in patients infected with C. difficule. The present example may also be extrapolated to non-CDI pathogens. Normally, patients must be exposed to the pathogen and become colonized via the fecal-oral route. This is facilitated by antibiotic use and in the case of C. difficule difficulty in killing spores; the patient's normal gut microbiota must be disturbed to allow pathogen invasion and proliferation, as is the case when antibiotics disrupt the normal intestinal microbiota ecosystem. C. difficule colonizes and expands within the host because they are antibiotic-resistant and can fill niches created by antimicrobial reduction of susceptible competitors. One can determine the extent to which patients become co-colonized by any antimicrobial resistant-pathogens and to characterize their emergence after hospital admission and subsequent role in infectious disease onset. Proliferating AMR-pathogens produce virulence factors and can disseminate e.g. C. difficule produces exotoxins that cause inflammation, colitis and diarrhea. The healthy microbiome plays an important role in preventing intestinal colonization and host susceptibility to AMR-pathogens. This is particularly noteworthy in recurrent CDI patients where fecal microbiota transplantation (FMT) provides highly effective clinical treatment in >90% of cases. The premise behind FMT therapy is re-establishment of a healthy gut microbiome in patients after pathogen clearance and in particular embodiments of the disclosure, gut microbiota health is a determinant in patient susceptibility to infection, a universally accepted concept in CDI. In any event, there needs to be a better understanding how different antibiotics modulate infection risk and subsequent morbidities via disruption of gut microbiota communities.

Embodiments of this disclosure combine highly synergistic metagenomics and metaproteomics data with extensive clinical outcomes expertise in the particular pathogens to perform in depth investigations of the pathogenic interplay between C. difficule, VRE and ESBL/CRE infection risk, the microbiota and the immunocompromised or critically ill patient.

One can characterize the functional microbiota features linked to C. difficule, VRE and ESNL/CRE infection risk and characterize common protective mechanisms against these AMR-pathogens. Data provided herein shows population-scale evidence that common protective microbiota features are missing in the most vulnerable patients, and there is demonstrated herein causation by identifying potent antimicrobials produced by these keystone microbiota species. This provides an opportunity for the characterization of the co-occurrence of these diverse pathogens. Recent functional metaproteomics data indicates that pathogen co-colonization and cross-talk may in fact be significantly underestimated when analyzed solely using metagenomics and, as such, requires an integrated systems approach to better understand how the metabolically active microbiota community functionally impacts clinical infectious disease susceptibility.

### *******************

Selection pressure driven by antibiotic overuse leads to new resistant pathogens that ultimately reduce drug efficacy. Identification of vulnerable patients and early detection of AMR-pathogens is critical when considering effective clinical management and minimizing the risk of emergent AMR traits. In specific embodiments, there is use of high risk clinical cohorts for longitudinal omics interrogation of functional host-microbiota-pathogen interactions that result in infectious disease susceptibility. Linking functional systems data with clinical phenotyping has not been performed in high risk patients who transition from pathogen colonization to symptomatic infection. The present omics data generated from adult and pediatric CDI cohorts shows that this type of investigative approach provides an unparalleled opportunity to predict infectious disease risk and mechanistically understand disease susceptibility at deep molecular and biochemical levels.

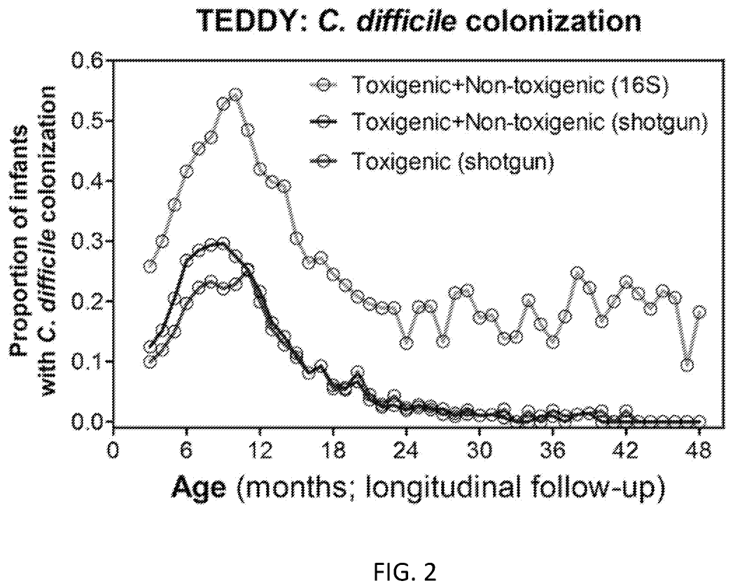

It is universally accepted that infants are highly susceptible to pathogen carriage and infectious disease progression. In infants, C. difficule colonization is common, with carrier rates up to 84%, which decrease to adult rates (˜3%) by 2-3 years of age (FIG. 2). The inventors' metagenomics exploration of microbiota features that contribute to disease susceptibility in pediatric and adult CDI patients found that C. difficule specifically targets individuals with infant-like gut microbiota features that we show are permissive to pathogen invasion and colonization. A core consortia of microbiota species was identified that show broad spectrum antimicrobial activity against C. difficule, VRE and ESBL/CRE, and confers protection against CDI in an infectious disease model. Based on the novel finding that defined keystone microbiota features are associated with CDI disease susceptibility, a new microbiome-based algorithm was generated that confidently predicts pathogen colonization resistance and CDI risk at a population-scale level. One can characterize these keystone microbiota species and identify their antimicrobial activity.

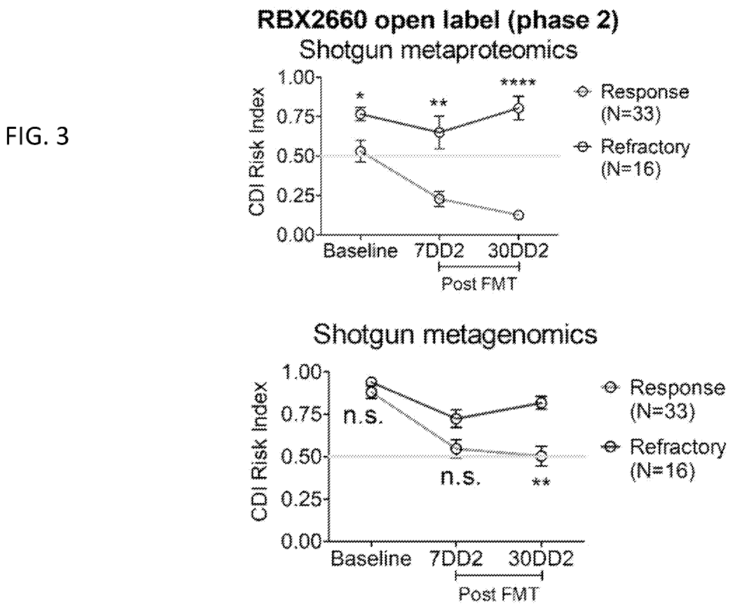

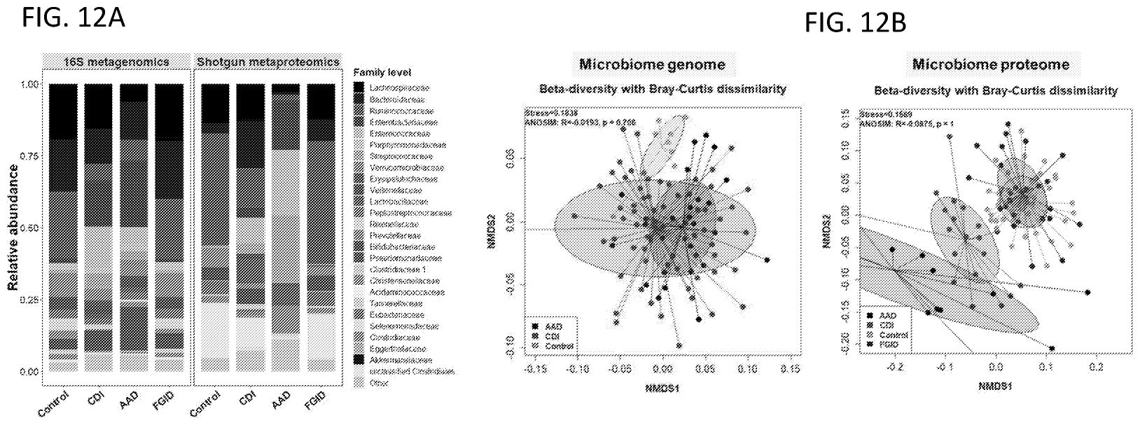

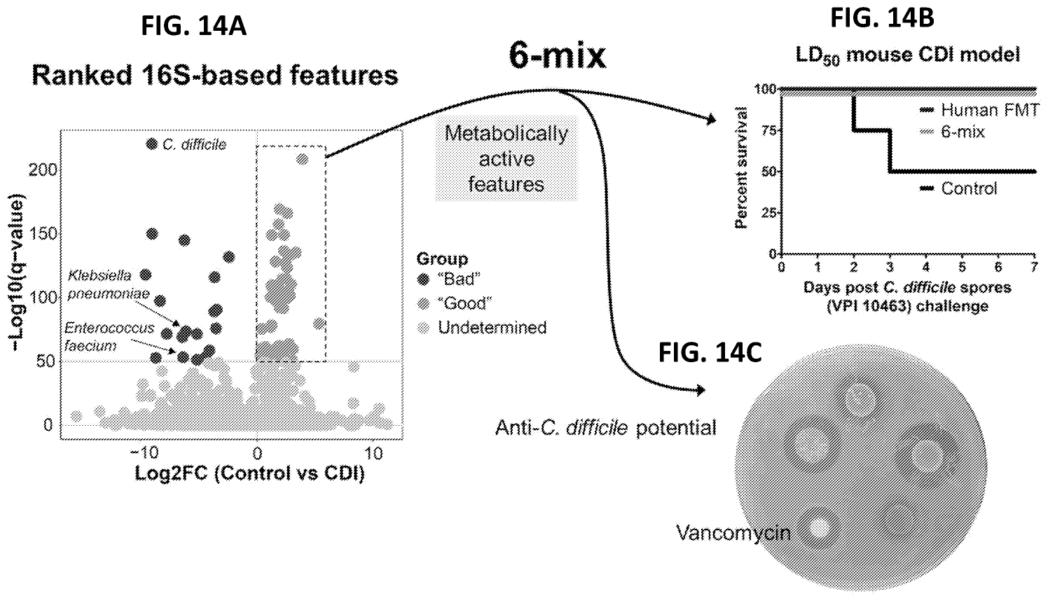

Despite the inventors' finding that metagenomics signatures can provide reliable microbiome-based classifiers of infectious disease susceptibility and clinical outcomes, functional validation studies are lacking. Using a high resolution shotgun metaproteomics platform, the inventors validated the importance of core microbiota features at the functional level and developed a new metaproteomics-based risk algorithm that enabled them to perform prototypical disease classification and clinical outcomes modeling that is not feasible using metagenomics data (FIGS. 3 and 12-14). Notably, they demonstrated that host-derived proteome interactions with the gut microbiota are powerful classifiers of infectious disease outcomes and there is provided new mechanistic insight as it relates to disease susceptibility in the critically ill and immunodeficient patient. This work is highly innovative and significant because it is generally assumed that the protective FMT mechanisms are microbial in nature and not due to host-derived protein signals. Furthermore, the inventors identified significant deviations in microbiome form and function when evaluating the inferred metagenome with its metabolically active counterpart in patients who are susceptible to infectious disease progression. Embodiments of the disclosure provide the development of metaproteome-based risk classifiers that identify patient susceptibility to CDI, VRE and ESBL/CRE infections, as shown herein using a microbiome-based approach. One can also mechanistically interrogate functional host-microbiota features that redefine the understanding of host-susceptibility to pathogens.

Encompassed herein is functional characterization of microbiota features and host-microbiota-pathogen interactions that are demonstrated to be significantly associated with intestinal colonization risk to multiple pathogens. Embodiments that utilize a metaproteomics analysis component are highly responsive to pathophysiologic conditions, making this omics approach ideally suited to distinguish subtle disease phenotypes that are not feasible using high resolution metagenomics alone. As encompassed herein, identification of host-microbiota classifiers that are highly predictive of clinical outcomes in infection allows one to integrate disease-associated pathways in the context of developing prototypical precision infection management strategies. Notably, a bioinformatics approach allows identification of patients in the general hospitalized population who are susceptible to infection and would benefit from precision infection management (e.g. contact isolation, FMT or prophylactic Bezlotoxumab), or antibiotic-avoidance in low-risk patients to manage development of disease susceptibility.

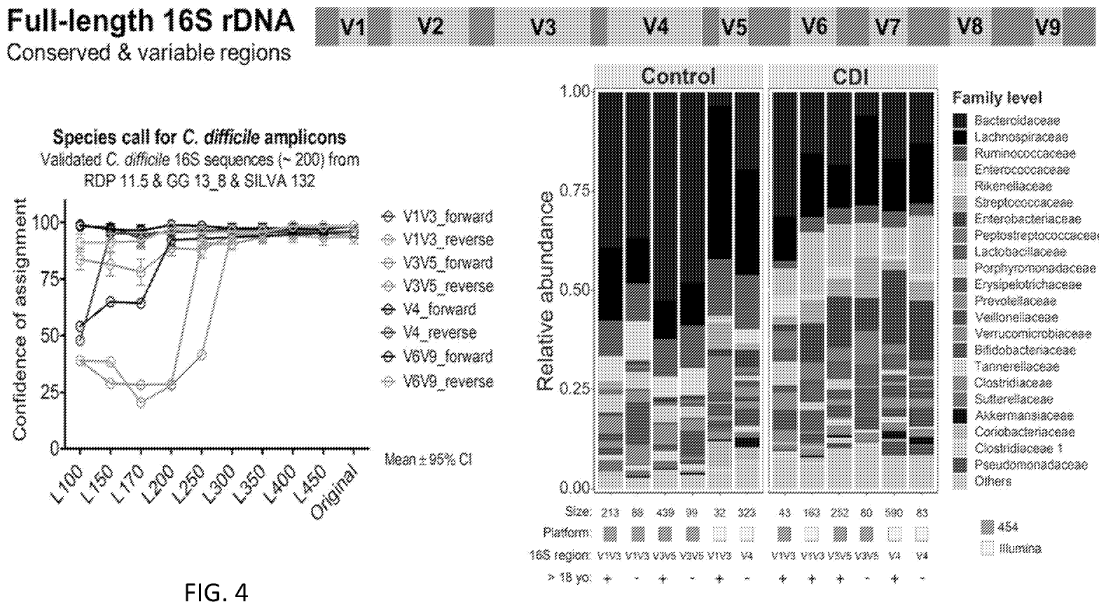

In one embodiment, the inventors incorporated 16S rDNA amplicon sequence data from multiple-center CDI trial sites (>1,200 adult and pediatric cases) as a larger combined analysis to reveal common microbiota features associated with CDI risk. These curated datasets define CDI-specific microbiome features for computational modelling and are sufficiently powered to account for demographic and geographic cohort variations, as well as providing the statistical rigor to exert confident disease-specific taxa association claims. Importantly, an analysis framework was developed allowing comparison of different 16S regions on different sequencing platforms and this bioinformatics approach was validated using (1) simulated 16S microbiome data, (2) C. difficule spiked fecal specimens, and (3) real-world CDI cohort datasets from Texas Medical Center institutions, including 16S microbiome data collected from non-diarrheal hospitalized controls and patients with CDI (primary or recurrent), antibiotic-associated diarrhea (AAD) and functional GI disorders (FGID or irritable bowel syndrome, IBS) as a disease control. These analyses demonstrated distinct microbiome features in CDI patients that can be confidently differentiated from healthy subjects or IBS patients who represent a common (<30%) CDI misdiagnosis (FIG. 4).

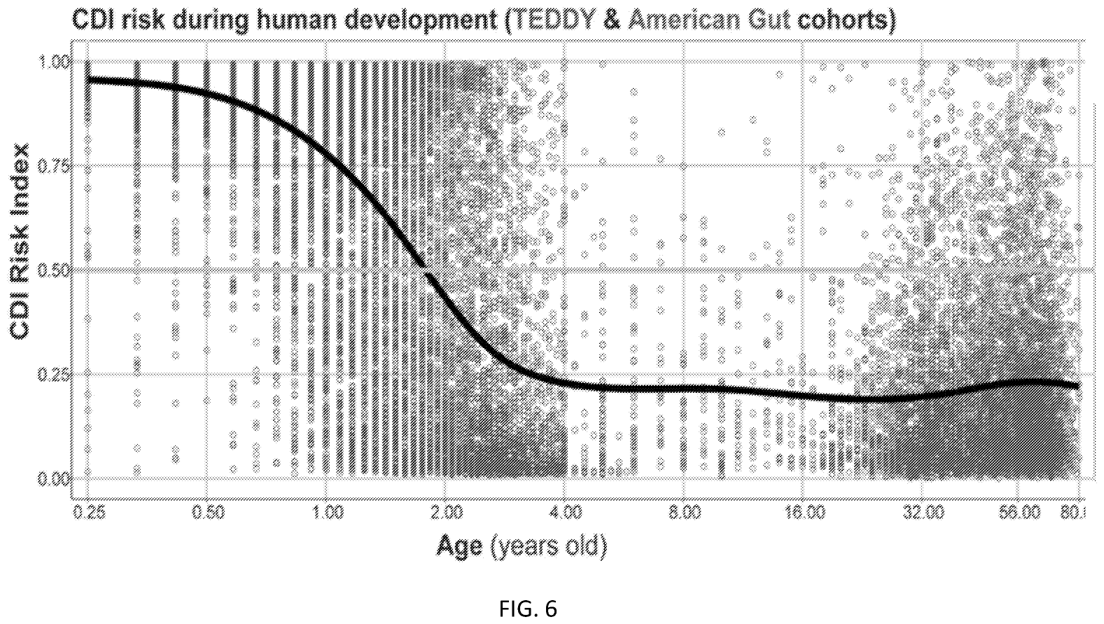

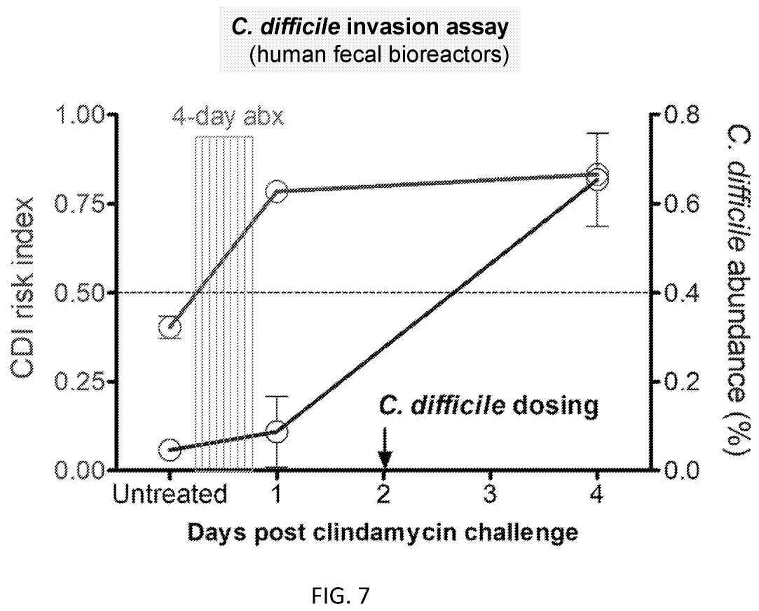

Supervised machine learning was utilized to identify the top 50 (as an example) discriminative microbiome features for CDI vs. hospitalized non-diarrheal controls or IBS disease controls using different algorithms. Those features after taxonomic binning at genus level built the most confident classification model with the Stacking learner providing a precision score≈0.95 and an AUC value >0.98 (FIG. 5). With a CDI recall classification accuracy >95% this algorithm performed significantly better in a side-by-side comparison of other reported microbiome risk indices in susceptible patients. To establish utility of the CDI risk algorithm, the inventors mined 16S microbiome data from several independent published cohorts providing population-scale evaluation of CDI risk in healthy individuals versus the general hospitalized population across the U.S: (1) American Gut Project and TEDDY microbiome sequencing archives of >15,000 healthy adult and pediatric subjects (FIGS. 6), and (2) patient cohorts (>5,000) with well-recognized clinical epidemiological data to support high, moderate and low CDI risk (FIG. 7). The metagenomics analysis confirmed the low CDI risk in the general U.S. population, unless subjects were either recently prescribed antibiotics or were young children (FIG. 6). In infants, asymptomatic C. difficule colonization is common, with carrier rates of up to 84% reported. Using TEDDY longitudinal infant study cohorts (N=900) located across the US and Europe we confirmed the high colonization rates of both toxigenic and non-toxigenic C. difficule and demonstrated a gradual parallel decrease in both CDI risk score and C. difficule colonzation with maturation of the gut microbiota during the first 3 years of life (FIGS. 2 and 6); 18 months appears to be the transition window from a microbiota that is permissive to C. difficule colonization to a healthy adult-like microbiota, although early antibiotic use in infants (mostly beta-lactams) delays this transition (data not shown). Although it is universally accepted that infants are highly susceptible to C. difficule colonization they do not generally develop clinical disease because they lack functionally active toxin receptors on the colonic mucosa that trigger inflammation. The inventors exploited these longitudinal findings in infants to provide independent validation of microbiome-features that are strongly associated with C. difficule colonization resistance during development. In strong support of the CDI risk algorithm, the inventors experimentally validated the model predictions by demonstrating that C. difficule invasion and colonization of complex microbiota communities in human fecal bioreactors accurately aligned (FIG. 7).

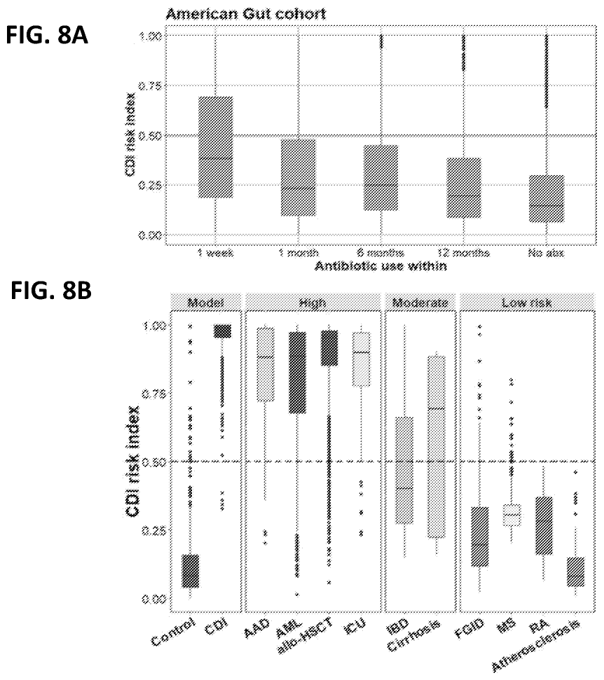

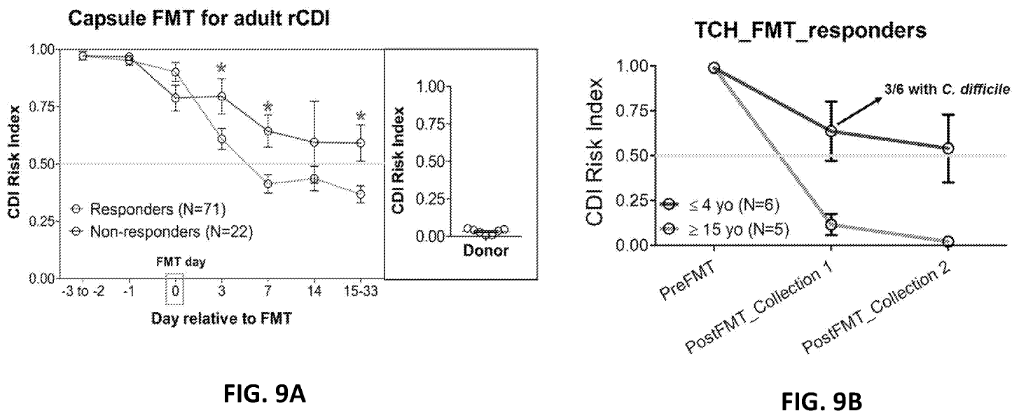

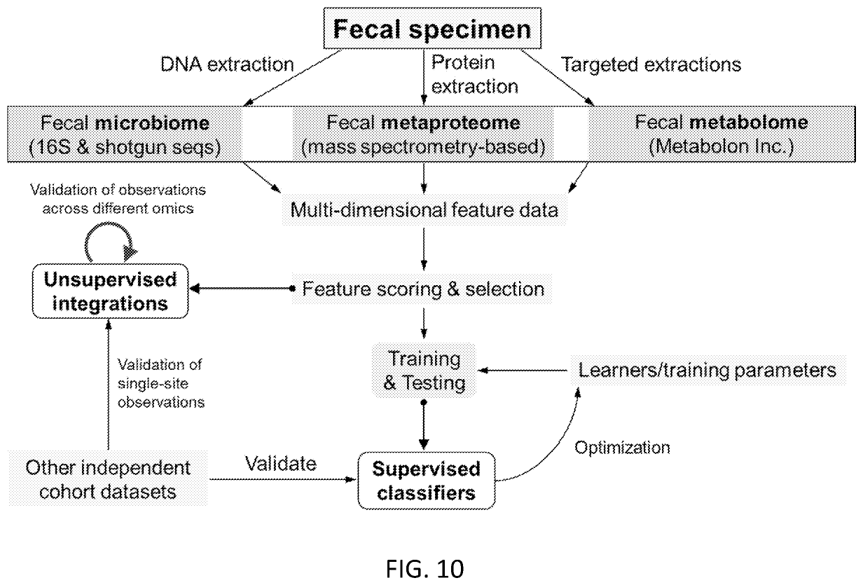

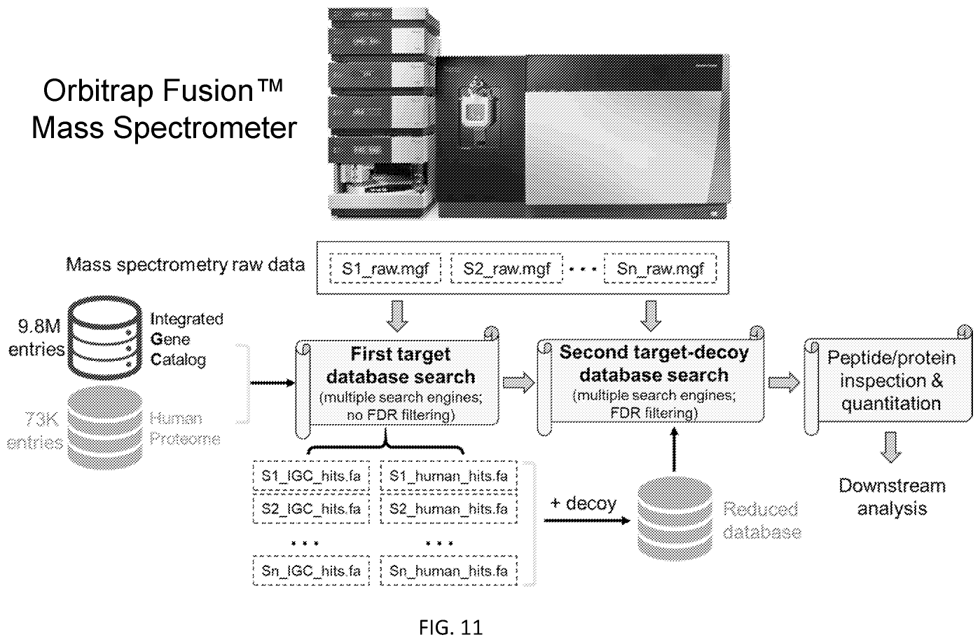

Predicting host susceptibility to NIAID-priority pathogens. With a CDI recall classification accuracy >95%, we mined 16S microbiome data from several independent published cohorts providing population-scale evaluation of CDI risk in the general hospitalized population using well characterized patient cohorts (>5,000 cases) with well described clinical epidemiological data to support high, moderate and low CDI risk (FIG. 8). Our analysis confirmed the low CDI risk in the general U.S. population, unless subjects were recently prescribed an antibiotic, the most significant risk factor for CDI (FIG. 8). As is well reported, CDI risk was demonstrated as high in asymptomatic C. difficule carriers, AAD and cancer patients at MD Anderson and Memorial Sloan Kettering, moderate in inflammatory bowel and liver disease, whereas it was low in cardiovascular disease and arthritis, which is in good agreement with the clinical epidemiology. We independently validated the classifier using CDI 16S sequencing data that was not part of our training set and demonstrated potential cases of CDI misdiagnosis, as well as excellent prediction of FMT outcomes in recurrent CDI patients (FIG. 10), although we have now improved clinical outcome predictions in FMT using metaproteome-based classifiers (FIG. 3). This work is significant because one can establish customized and precision health metagenomics approaches for precision-based diagnosis and management of CDI, VRE and ESBL-E/CRE risk as a novel infection control strategy.

Bioinformatics Analysis of Shotgun Metaproteome Data

Mass spectrometry output files generated from label-free proteomic workflow were converted into mascot generic format (MGF) files by msConvert from ProteoWizard (version 3.0.18240) for downstream processing with the strategy of two-step database search. Human protein sequences from UniProt database and microbial protein sequences from comprehensive, non-redundant Integrated Gene Catalog (IGC) database of human gut microbiome (known and uncultured microbes) were download from respective public repositories as the target database. The first target search for MGF files was performed by SearchGUl (version 3.3.3) applying X!Tandem search engine without false discovery rate (FDR) filtering. Unique protein hits from first step search were extracted from human and IGC databases as reduced target database; decoy database was generated by reversing the reduced target sequences. Protein sequence of trypsin (used for digestion) of specific origin was included to the concatenated reduced target-decoy database. The second target-decoy search was performed for all MGF files with the above reduced database by SearchGUl applying X!Tandem with FDR score of 0.01. Second search results were further inspected and interpreted by PeptideShaker (version 1.16.40). Confident protein hits with at least two unique peptides identified were included for downstream analysis. Taxonomic assignment for the sequences of IGC protein hits (only main accession) was achieved by using lowest common ancestor algorithm for interpreting diamond (version 0.9.22.123) searches against NCBI NR database (downloaded in January 2019). In general, spectral counting metric (similar to the terms—contig coverage & gene abundance in shotgun metagenomic analyses) outperforms peak intensity in terms of biological interpretation of gut microbiome studies. Thus spectral counts, generated from PeptideShaker employing protein inference coefficient-weighted Normalized Spectral Abundance Factor (NSAF), were used for calculating taxonomic composition based on the collapsed taxonomies (from species to phylum rank) of IGC protein hits within one sample.

### ******************************************************************

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of determining a cause of diarrhea in an individual comprising measuring for one or more features encompassed in the disclosure herein present in a gut sample from the individual.

2. The method of claim 1, wherein the gut sample is a fecal sample.

3. The method of claim 1, further comprising modulating a treatment for the individual determined to have one or more feature levels that indicate the presence or absence of one or more diarrheal-associated diseases.

4. The method of claim 3, further comprising administering a treatment or reducing a treatment to the individual when the individual is determined to have one or more feature levels that indicate the presence or absence of one or more diarrheal-associated diseases.

5. The method of claim 1, wherein the individual having one or more features encompassed in the disclosure herein is determined to have a pathogenic infection.

6. The method of claim 5, wherein the pathogen is a bacteria, virus, parasite, fungus, or mixture thereof.

7. The method of claim 5, wherein the pathogen is Corynebacterium; Enterococcus faecium; Enterococcus; Escherichia coli; Fungal pneumonia; Klebsiella; Pseudomonas aeruginosa; Staphylococcus aureus (MRSA); Stenotrophomonas pneumonia; Streptococcus pneumonia; Vancomycin-resistant Enterococcus, or a mixture thereof.

8. The method of claim 5, wherein the pathogen is Campylobacter (jejuni, coli and/or upsaliensis); C. difficule; Plesiomonas shigelloides; Salmonella; Yersinia enterocolitica; Vibrio (parahaemolyticus, vulnificus and/or cholerae); diarrheagenic E. coli/Shigella (enteroaggregative E. coli [EAEC]; enteropathogenic E. coli [EPEC]; enterotoxigenic E. coli [ETEC]; Shiga toxin-producing E. coli [STEC] O157; Shigella/Enteroinvasive E. coli [EIEC]); Cryptosporidium; Cyclospora cayetanensis; Entamoeba histolytica; Giardia lamblia; rotavirus A; adenovirus F 40/41; astrovirus; norovirus G1/GII; sapovirus I, II, IV, and/or V.

9. The method of claim 5, wherein the pathogen is Clostridioides.

10. The method of claim 9, wherein the Clostridioides is Clostridioides difficule, Clostridioides perfingens, Clostridioides botulinum, or a mixture thereof.

11. The method of claim 1, wherein the individual having one or more features is determined to have antibiotic associated diarrhea.

12. The method of claim 1, wherein the measuring identifies the presence or absence of one or more features encompassed in the disclosure herein.

13. The method of claim 1, wherein the measuring identifies a level of one or more features encompassed in the disclosure herein.

14. The method of claim 13, wherein the level of one or more features is compared to a threshold or known standard.

15. The method of claim 1, wherein the individual is an adult, child, or infant.

16. The method of claim 1, wherein the individual has recurrent diarrhea.

17. The method of claim 1, wherein the individual is suspected of having misdiagnosis of a cause for the diarrhea.

18. A method of treating an individual having diarrhea comprising
measuring for one or more features encompassed herein from a fecal sample from the individual; and
reducing the administration of antibiotics and/or antimicrobial treatment to the individual when the individual has presence or absence or a certain level of one or more feature(s) indicative of antibiotic associated diarrhea; or
administering antibiotics and/or antimicrobial treatment to the individual when the individual has presence or absence or a certain level of one or more feature(s) indicative of pathogenic infection.

19. A method of treating an individual having diarrhea comprising
measuring for one or more features encompassed herein from a fecal sample from the individual; and
reducing the administration of antibiotics and/or antimicrobial treatment for an individual determined to have the presence or absence or a certain level of one or more feature(s) indicative of antibiotic associated diarrhea; or
administering antibiotics and/or antimicrobial treatment to an individual determined to have the presence or absence or a certain level of one or more feature(s) indicative of pathogenic infection.

20. The method of claim 18, wherein the antibiotics comprise at least one of the antibiotics selected from the group consisting of a small molecule antibiotic, an antibiotic derived from a natural product, a microbial composition, an antibody suitable for neutralizing pathogenic infections, a therapeutic, contact isolation, and a combination thereof.

21. The method of claim 18, wherein the pathogen is C. difficule.

22. A method of measuring one or more features encompassed herein in a fecal or gut sample from an individual that has diarrhea, that has recurrent diarrhea, and/or that is suspected of having a misdiagnosis of a diarrheal cause, comprising the steps of two or more of the following:
analyzing one or more nucleic acids in the sample;
analyzing one or more metabolites in the sample; and
analyzing one or more proteins in the sample.

23. The method of claim 22, wherein the analyzing steps include one or more features encompassed herein.

24. The method of claim 22, wherein the nucleic acid is analyzed by sequencing, polymerase chain reaction, isothermal amplification, bioinformatics, or a combination thereof.

25. The method of claim 22, wherein the metabolites are analyzed by mass spectrometry, ELISA, chromatography, or a combination thereof.

26. The method of claim 22, wherein the proteins are analyzed by mass spectrometry, ELISA, chromatography, Western blotting, immunoprecipitation, immunoelectrophoresis, or a combination thereof.

27. The method of claim 22, wherein the nucleic acid analyzed is 16S ribosomal RNA.

28. A method to measure a host response to a microbial infection in an individual, said individual that has diarrhea, that has recurrent diarrhea, and/or that is suspected of having a misdiagnosis of a diarrheal cause, comprising the steps of
analyzing one or more nucleic acids in a fecal or gut sample from the individual;
analyzing metabolites in the sample; and/or
analyzing proteins in the sample.

29. The method of claim 28, wherein the analyzing steps include one or more features of any one of Tables A-C.

30. The method of claim 28, wherein the nucleic acid is analyzed by sequencing, polymerase chain reaction, isothermal amplification, bioinformatics, or a combination thereof.

31. The method of claim 28, wherein the metabolites are analyzed by mass spectrometry, ELISA, chromatography, or a combination thereof.

32. The method of claim 28, wherein the proteins are analyzed by mass spectrometry, ELISA, chromatography, Western blotting, immunoprecipitation, immunoelectrophoresis, or a combination thereof.

33. The method of claim 28, wherein the nucleic acid analyzed is 16S ribosomal RNA.

34. The method of claim 1, wherein the feature is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 1443, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 82, 183, 184, 185, 186, 187, 188, 189, 190, 191, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, or more features of any one of Tables A-C.

35. The method of claim 1, wherein the feature is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of the features of any one of Tables A-C.
